(19) United States
(12) Patent Application Publication
(10) Pub. No.: US 2025/0145715 A1
(43) Pub. Date: May 8, 2025

(54) B7-H3 TARGETING FUSION PROTEINS AND METHODS OF USE THEREOF

(71) Applicant: GT Biopharma, Inc., Brisbane, CA (US)

(72) Inventor: Martin Schroeder, Brisbane, CA (US)

(21) Appl. No.: 18/690,991

(22) PCT Filed: Sep. 15, 2022
(86) PCT No.: PCT/US2022/043708

§ 371 Date: Mar. 11, 2024

Related U.S. Application Data
(60) Provisional application No. 63/245,132, filed on Sep. 16, 2021.

(51) Int. Cl. C07K 16/28; A61K 39/00; A61P 35/00; C07K 14/54

(52) U.S. Cl. CPC C07K 16/2827; A61P 35/00; C07K 14/5443; C07K 16/283; A61K 2039/505; C07K 2317/22; C07K 2317/24; C07K 2317/31; C07K 2317/569; C07K 2317/622; C07K 2317/732; C07K 2319/30

(57) ABSTRACT

The invention provides B7-H3 targeting fusion proteins and methods of use thereof. The targeting fusion proteins include B7-H3 targeting tri-specific killer engager molecules comprising a B7-H3 targeting binding protein, a CD16 targeting binding protein, and an interleukin-15 protein. The methods of use thereof include methods of treating cancer, methods of inducing natural killer (NK) cell activity against a cancer cell, methods of inhibiting tumor growth, methods of increasing survival of a subject having cancer, and methods of inducing NK-mediated antibody-dependent cellular cytotoxicity against a cancer cell in a subject.

### CROSS-REFERENCE TO RELATED APPLICATIONS

This application claims benefit of priority under 35 U.S.C. § 119(e) of U.S. Provisional Application No. 63/245,132, filed Sep. 16, 2021. The disclosure of the prior application is considered part of and are herein incorporated by reference in the disclosure of this application in its entirety.

### INCORPORATION OF SEQUENCE LISTING

The material in the accompanying sequence listing is hereby incorporated by reference into this application. The accompanying sequence listing xml file, name G1421US00_GTBIO2180-1WO.xml, was created on Sep. 13, 2022 and is 55 kb in size.

### BACKGROUND OF THE INVENTION

### Field of the Invention

The present invention relates generally to fusion proteins, and more specifically to B7-H3 targeting tri-specific killer engager molecules and their use to treat cancer.

### Background Information

Immunotherapy is an individualized treatment that activates or suppresses the immune system to amplify or diminish an immune response and is developing rapidly for treating various forms of cancer. Immunotherapy for cancer, such as chimeric antigen receptor (CAR)-T cells, CAR-natural killer (NK) cells, PD-1 and PD-L1 inhibitor, aims to help patients' immune system fight cancer. The activation of T cell depends on both the specific combination of T cell receptor (TCR) and peptide-bound major histocompatibility complex (MHC), and the interplay of co-stimulatory molecules of T cell with ligands on antigen presenting cells (APCs). The B7 families, peripheral membrane proteins on activated APCs, have been shown to participate in regulation of T cell responses. Recent studies indicate that the upregulation of inhibitory B7 molecules in the cancer microenvironment is highly related to the immune evasion of tumor. As a newly identified member of the B7 family, B7-H3 could promote the activation of T cells and the production of IFN-γ.

Different B7 molecules have either positive or negative co-stimulatory signals while modulating immune cell responses. Immune checkpoints, such as PD-1, PD-L1, PD-L2, and CTLA4, are molecules holding many receptor-ligand interactions to evade the immune system and facilitate proliferation. Several monoclonal antibodies (mAbs) that block these proteins were developed to down-regulate the inhibitory immune response and promote the cellular cytotoxicity of T cells that eliminate tumor cells. Among the immune checkpoint-blocking drugs, the inhibitors targeting PD-1 or CTLA4 were successfully used for treating patients with metastatic melanoma, with improved responses and prolonged survival. This success led to the development of such agents for treating a wide range of malignancies, including renal cell carcinoma (RCC), NSCLC, and acute myeloid leukemia (AML), which further enhanced the response rate compared to conventional treatments, and prolonged the survival time of patients (Yang et al., Int J Biol Sci 2020; 16(11):1767-1773).

B7-H3 was found to be overexpressed among several kinds of human cancer cells and was correlated with disease deteriorations. B7-H3 was recognized as a co-stimulatory molecule for immune reactions such as T cell activation and IFN-7 production. In the presence of anti-CD3 antibody mimicking the TCR signal, human B7-H3-Ig fusion protein increases the proliferation of both CD4+ and CD8+ T cells and enhances the cytotoxic T lymphocyte (CTL) activity in vitro. B7-H3 also has an antitumor effect on adenocarcinoma of the colon, which could also be regarded as a promising therapy for the treatment of colon cancers. In a study among human pancreatic cancer patients, B7-H3 was recognized as a co-stimulatory molecule that was not only abundantly expressed in pancreatic cancer but also associated with increased treatment efficacy. Although B7-H3 expression was detectable in most examined pancreatic cancer samples, and significantly upregulated in pancreatic cancer versus normal pancreas, patients with high tumor B7-H3 levels had a significantly better postoperative prognosis than patients with low tumor B7-H3 levels (Yang et al., ibid).

Despite certain successes, there are limitations that decrease the overall efficiency of mAb therapies. With the development of CD16-directed bispecific and tri-specific single-chain fragment variable (BiKEs and TriKEs) recombinant molecules, most of these undesired limitations are avoided while eliciting high effector function as they lack the Fc portion of whole antibodies and have a targeted specificity for CD16 (Gleason et al., Mol Cancer Ther; 11(12); 2674-84, 2012). As a result, recombinant reagents are attractive for clinical use in enhancing natural killer (NK) cell immunotherapies.

The ability of NK cells to recognize and kill targets is regulated by a sophisticated repertoire of inhibitory and activating cell surface receptors. NK cell cytotoxicity can occur by natural cytotoxicity, mediated via the natural cytotoxicity receptors (NCR), or by antibodies, such as rituximab, to trigger antibody-dependent cell-mediated cytotoxicity (ADCC) through CD16, the activating low-affinity Fc-7 receptor for immunoglobulin G (IgG) highly expressed by the CD56^{dim} subset of NK cells. CD16/CD19 BiKE and CD16/CD19/CD22 TriKE can trigger NK cell activation through direct signaling of CD16 and induce directed secretion of lytic granules and target cell death. Furthermore, these reagents induce NK cell activation that leads to cytokine and chemokine production.

### SUMMARY OF THE INVENTION

The present invention is based on the development of B3-H7 targeting fusion proteins, and specifically B7-H3 targeting tri-specific killer engager molecules (TriKEs) and on methods of use thereof.

In one embodiment, the present invention provides an isolated nucleic acid sequence as set forth in SEQ ID NO:13 or 14 or a sequence having 90% identity thereto.

In another embodiment, the invention provides a protein encoded by a nucleic acid sequence as set forth in SEQ ID NO:13 or 14 or a sequence having 90% identity thereto.

In one aspect, the amino acid sequence is selected from SEQ ID NO:6 or 7.

In an additional embodiment, the invention provides a fusion protein including the amino acid sequence set forth in SEQ ID NO:6 and 7, operably linked to each other in either orientation.

In one aspect, the protein includes SEQ ID NO:6 and 7, in direct linkage between the C-terminus of SEQ ID NO:6 and the N-terminus of SEQ ID NO:7. In another aspect, the protein includes SEQ ID NO:7 and 6, in direct linkage between the C-terminus of SEQ ID NO:7 and the N-terminus of SEQ ID NO:6.

In a further embodiment, the invention provides a fusion protein including the sequence set forth in SEQ ID NO:1 and sequences having 90% or greater identity to SEQ ID NO:1.

In one embodiment, the invention provides a fusion protein including in operably linkage, SEQ ID NO:2 or 19; 4, 17, or 18; 6 and 7, or 7 and 6.

In one aspect, SEQ ID NO:2 or 19 and 4, 17 or 18 are linked by SEQ ID NO:3 or SEQ ID NO:15. In another aspect, SEQ ID NO:4, 17 or 18 and 6 or 7 are linked by SEQ ID NO:5 or SEQ ID NO:16. In other aspects, SEQ ID NO:6 and 7 are in operable linkage in either orientation. In some aspects, the fusion protein further includes a half-life extending (HLE) molecule. In one aspect, the HLE molecule is a Fc or a scFc antibody fragment including any one of SEQ ID NOs:21-25. In some aspects, SEQ ID NO:4 has an N72 substitution. In various aspects, the N72 mutation is N72A or N72D, set forth in SEQ ID NO:17 and 18, respectively.

In an additional embodiment, the invention provides an isolated nucleic acid sequence encoding any of the fusion proteins described herein.

In one aspect, the sequence is SEQ ID NO:8.

In another embodiment, the invention provides a method of treating cancer in a subject including administering to the subject any of the fusion proteins described herein, thereby treating the cancer.

In one aspect, the cancer is selected from non-small lung cancer, cutaneous squamous cell carcinoma, pancreatic cancer, primary hepatocellular carcinoma, colorectal carcinoma, clear cell renal carcinoma or breast cancer.

In an additional embodiment, the invention provides a fusion protein comprising in operable linkage, SEQ ID NO:2, SEQ ID NO:4 and SEQ ID NO:6 and 7 in either orientation or SEQ ID NO:19, SEQ ID NO:17 or 18 and SEQ ID NO:6 and 7 in either orientation and nucleic acid sequences encoding such proteins.

In one aspect, SEQ ID NO:19 is operably linked to SEQ ID NO:17 or 18 by a linker of SEQ ID NO:3 or 15. In another aspect, SEQ ID NO:17 or 18 is operably linked to SEQ 6 and 7, in either orientation, by a linker of SEQ ID NO:5 or 16. In some aspects, the fusion protein further includes a half-life extending (HLE) molecule. In one aspect, the HLE molecule is a Fc or a scFc antibody fragment including any one of SEQ ID NOs:21-25.

In one embodiment, the invention provides a pharmaceutical composition including a therapeutically effective amount of a fusion protein including the amino acid sequence of SEQ ID NO:1 or a sequence having 90% or greater identity to SEQ ID NO:1 and a pharmaceutically acceptable carrier.

In another embodiment, the invention provides a method of treating cancer in a subject including administering to the subject the pharmaceutical composition described herein.

In an additional embodiment, the invention provides a method of inducing natural killer (NK) cell activity against a cancer cell in a subject including administering to the subject a fusion protein including the sequence set forth in SEQ ID NO:1 and sequences having 90% or greater identity to SEQ ID NO:1, thereby inducing NK cell activity against a cancer cell in the subject.

In one aspect, inducing NK cell activity includes inducing NK cells degranulation, inducing NK cell production of interferon 7, increasing a number of tumor infiltrating NK cells in the subject, and/or inducing or increasing NK cell proliferation.

In one embodiment, the invention provides a method of inhibiting tumor growth in a subject including administering to the subject a fusion protein including the sequence set forth in SEQ ID NO:1 and sequences having 90% or greater identity to SEQ ID NO:1, thereby inhibiting tumor growth in the subject.

In one aspect, inhibiting tumor growth includes decreasing tumor cell survival.

In another embodiment, the invention provides a method of increasing survival of a subject having cancer including administering to the subject a fusion protein including the sequence set forth in SEQ ID NO:1 and sequences having 90% or greater identity to SEQ ID NO:1, thereby increasing survival of the subject.

In an additional embodiment, the invention provides a method of inducing natural killer (NK) mediated antibody-dependent cellular cytotoxicity against a cancer cell in a subject including administering to the subject a fusion protein including the sequence set forth in SEQ ID NO:1 and sequences having 90% or greater identity to SEQ ID NO:1, thereby increasing survival of the subject.

In one aspect, administering to a subject a fusion protein including the sequence set forth in SEQ ID NO:1 and sequences having 90% or greater identity to SEQ ID NO:1 further includes administering to the subject an anti-cancer treatment.

In another aspect, the subject has cancer. In some aspects, the cancer is selected from the group consisting of lung cancer, prostate cancer, multiple myeloma, ovarian cancer and head and neck cancer. In other aspects, cancer cells are B7-H3 expressing cancer cells. In some aspects, the cancer is a treatment resistant cancer.

### BRIEF DESCRIPTION OF THE DRAWINGS

FIGS. 1A-1D illustrate the construction and isolation of cam1615B7-H3 tri-specific killer engager (TriKE). FIG. 1A is a schematic representation of the TriKE construct consisting of (left to right) camelid anti-CD16 VHH, Human IL-15, and anti-B7-H3 scFv. FIG. 1B is a graph illustrating the chromatography trace from the first-step purification of cam1615B7-H3 on an ion exchange (FFQ) column. The collection peak is indicated by the double-sided arrow. FIG. 1C is a graph illustrating the chromatography trace from the second-step purification of cam1615B7-H3 on a size exclusion chromatography (SEC) column. The collection peak is indicated by the double-sided arrow. FIG. 1D is a photograph of a sodium dodecyl sulfate polyacrylamide gel electrophoresis (SDS-PAGE) gel indicating the purity of the final product after the two orthogonal column steps. The lanes of the gel display molecular marker, non-reduced (NR) product, and reduced (R) product.

FIGS. 2A-2H illustrates that cam1615B7-H3 TriKE induces potent, and specific, natural killer (NK) cell proliferation. Peripheral blood mononuclear cells (PBMCs) were CellTrace Violet labeled and incubated for seven days at 5 nM equimolar concentrations of rhIL-15 or cam1615B7-H3 TriKE. On day 7, the cells were harvested and stained for flow cytometric evaluation. FIG. 2A shows a representative histogram showing NK cell (CD56+CD3−) proliferation measured as the dilution of the CellTrace Violet dye. FIG. 2B is a graph illustrating pooled data showing the overall proportion of NK cells that proliferated. FIG. 2C is a graph illustrating pooled data showing the proportion of NK cells that highly proliferated (measured as proliferation beyond three divisions). FIG. 2D is a graph illustrating pooled data showing NK cell counts in the cultures (N=9). FIG. 2E is a representative histogram showing T cell (CD56− CD3+) proliferation. FIG. 2F is a graph illustrating pooled data showing the overall proportion of T cells proliferated. FIG. 2G is a graph illustrating pooled data showing the proportion of T cells highly proliferated. FIG. 2H is a graph illustrating pooled data showing T cell counts in the cultures (N=9). * p<0.05, ** p<0.01, *** p<0.001.

FIGS. 3A-3B illustrate camB7-H3 TriKE binding specificity. FIG. 3A is a schematic representation of the binding of the TriKE molecule to B7-H3 positive cancer cells. FIG. 3B is a graph illustrating the binding specificity to WT B7-H3, BT-12 pediatric brain tumor lines highly express B7-H3 (WT) while B7-H3 KO BT-12 (red) cell line was produced using CRISPR.

FIGS. 4A-4B illustrates the ADCC induction by the TriKE molecules. FIG. 4A is a graph showing the ADCC induction of the WT TriKE. FIG. 4B is a graph showing the ADCC induction of the CD16 TriKE.

FIGS. 5A-5B illustrate functional assays conducted with hematologic malignancy cell lines with varying levels of B7-H3 expression from none to very high levels. FIG. 5A is a graph illustrating NK cell activation measured using CD107a as measured by flow cytometry (n=3), IncuCyte, or xCelligence assay. FIG. 5B is a graph illustrating NK cell activation measured using IFN gamma as measured by flow cytometry (n=3), IncuCyte, or xCelligence assay.

FIGS. 6A-6B illustrates B7-H3 MFI on four myeloma cell lines by flow cytometry. FIG. 6A is a graph showing the expression of B7-H3 on the myeloma lines RPMI-8226, U266, MM1S and H929 by flow cytometry. FIG. 6B is a graph summarizing the data in FIG. 6A.

FIGS. 7A-7B illustrate the ability of peripheral blood NK cells with or without B7-H3-TriKE to kill myeloma cells in live imaging IncuCyte Zoom assays. FIG. 7A is a graph showing the effects with effector:target (E:T) ratios of 2:1 and 4:1 on MM1S cells. FIG. 7B is a graph showing the effects with effector:target (E:T) ratios of 2:1 and 4:1 on U266 cells.

FIGS. 8A-8D illustrate the ability of peripheral blood NK cells with or without B7-H3-TriKE to kill myeloma cells in live imaging IncuCyte Zoom assays. FIG. 8A is a graph showing the effects with effector:target (E:T) ratios of 2:1 and 4:1 on H929 cells. FIG. 8B is a graph showing the effects with effector:target (E:T) ratios of 2:1 and 4:1 on MM1S cells. FIG. 8C is a graph showing the effects with effector:target (E:T) ratios of 2:1 and 4:1 on RPMI-8226 cells. FIG. 8D is a graph showing the effects with effector:target (E:T) ratios of 2:1 and 4:1 on U266 cells.

FIGS. 9A-9D illustrate the efficacy of B7-H3-TriKE with the proteasome inhibitor bortezomib (10 nM) and the immunomodulatory drug lenalidomide (5 μM). FIG. 9A is a graph showing the effect of the combination therapy on H929 cells after 48 hours. FIG. 9B is a graph showing the effect of the combination therapy on RPMI-8226 cells after 48 hours. FIG. 9C is a graph showing the effect of the combination therapy on MM1S cells after 48 hours. FIG. 9D is a graph showing the effect of the combination therapy on U266 cells after 48 hours.

FIGS. 10A-10D illustrate the effect B7-H3-TriKE on MDSC developed from CD33+ myeloid cells from healthy donors, when incubated with myeloma cells at 1:100 ratio. FIG. 10A is a graph showing MDSC (CD14+CD11b+) expression of B7-H3. FIG. 10B is a graph showing cell survival as measured by flow cytometry. FIG. 10C is a plot of live, CD14+ cells FIG. 10D is a graph illustrating H929 growth as measured over 48 hours by live cell imaging.

FIGS. 11A-11B illustrate NK mediated killing of B7-H3 expressing MDSC. FIG. 11A is a graph showing B7-H3 expression of MDSC. FIG. 11B is a graph illustrating NK mediated killing of co-cultured MDSC with NK at E:T of 1:1 and compared killing with and without B7-H3 TriKE.

FIGS. 12A-12D show that cam1615B7-H3 TriKE enhances NK cell function against prostate tumor targets. FIG. 12A is a graph illustrating pooled proportion of NK cells from healthy donors expressing CD107a+ (degranulation) against CREB5, 22RV1 and Enza-R targets. FIG. 12B is a graph illustrating pooled proportion of NK cells from healthy donors expressing IFNγ cytokine production against CREB5, 22RV1 and Enza-R targets. FIG. 12C is a graph illustrating pooled proportion of NK cells from healthy donors expressing CD107a+ (degranulation). FIG. 12D is a graph illustrating pooled proportion of NK cells from healthy donors expressing IFNγ cytokine production.

FIGS. 13A-13B show that camB7-H3 increase NK cell function against prostate cancer cells and NK proliferation as compared to IL-15 Alone. FIG. 13A is a graph showing percent of NK cells expressing IFNγ against prostate cancer cell targets. FIG. 13B is a graph illustrating the proportion of NK cells undergoing 3 or more rounds of division.

FIGS. 14A-14L show that TriKEs can induce NK cell degranulation and inflammatory cytokine production against prostate cancer cell lines. FIG. 14A is a graph showing pooled analysis of proportion of NK cells from healthy donors or prostate cancer patients expressing CD107a+ against CA-2 targets. FIG. 14B is a graph showing pooled analysis of proportion of NK cells from healthy donors or prostate cancer patients expressing CD107a+ against PC-3 targets. FIG. 14C is a graph showing pooled analysis of proportion of NK cells from healthy donors or prostate cancer patients expressing CD107a+ against DU-145 targets. FIG. 14D is a graph showing pooled analysis of proportion of NK cells from healthy donors or prostate cancer patients expressing IFNγ against CA-2 targets. FIG. 14E is a graph showing pooled analysis of proportion of NK cells from healthy donors or prostate cancer patients expressing IFNγ against PC-3 targets. FIG. 14F is a graph showing pooled analysis of proportion of NK cells from healthy donors or prostate cancer patients expressing IFNγ against DU-145 targets. FIG. 14G is a graph showing pooled analysis of proportion of NK cells from healthy donors or prostate cancer patients expressing CD107a+ against LnCAP targets. FIG. 14H is a graph showing pooled analysis of proportion of NK cells from healthy donors or prostate cancer patients expressing CD107a+ against VCAP targets. FIG. 14I is a graph showing pooled analysis of proportion of NK cells from healthy donors or prostate cancer patients expressing CD107a+ against 22RV1 targets. FIG. 14J is a graph showing pooled analysis of proportion of NK cells from healthy donors or prostate cancer patients expressing IFNγ against LnCAP targets. FIG. 14K is a graph showing pooled analysis of proportion of NK cells from healthy donors or prostate cancer patients expressing IFNγ against VCAP targets. FIG. 14L is a graph showing pooled analysis of proportion of NK cells from healthy donors or prostate cancer patients expressing IFNγ against 22RV1 targets.

FIGS. 15A-15D show that cam1615B7-H3 TriKE enhances NK cell function of healthy donors (black bars) and prostate cancer patients (white bars) against prostate tumor targets. FIG. 15A is a graph showing pooled analysis of proportion of NK cells from healthy donors or prostate cancer patients expressing CD107a+ (top) or IFNγ (bottom) against C4-2 targets (N=9 for healthy donors, N=3 for patients). FIG. 15B is a graph showing pooled analysis of proportion of NK cells from healthy donors or prostate cancer patients expressing CD107a+ (top) or IFNγ (bottom) against DU145 targets (N=9 for healthy donors, N=3 for patients). FIG. 15C is a graph showing pooled analysis of proportion of NK cells from healthy donors or prostate cancer patients expressing CD107a+ (top) or IFNγ (bottom) against LNCaP targets (N=9 for healthy donors, N=3 for patients). FIG. 15D is a graph showing pooled analysis of proportion of NK cells from healthy donors or prostate cancer patients expressing CD107a+ (top) or IFNγ (bottom) against PC3 targets (N=9 for healthy donors, N=3 for patients). * p<0.05, ** p<0.01, *** p<0.001, and **** p<0.0001.

FIG. 16 is a graph illustrating a PC3 standard IncuCyte Assay.

FIG. 17 shows photographs illustrating PC-3 spheroid under various treatment conditions.

FIG. 18 is a graph illustrating the size of PC-3 spheroids over time.

FIG. 19 is a graph illustrating the size of PC-3 spheroids over time.

FIG. 20 shows photographs illustrating the ability of B7-H3 TriKE and BiKE to mediate efficient killing of PC3 spheroids.

FIG. 21 is a graph illustrating cell index over time.

FIGS. 22A-22F illustrate enzalutamide resistant prostate cancer cell line phenotyping for B7-H3. FIG. 22A is a graph illustrating B7-H3 expression in CREB5+ cells. FIG. 22B is a graph illustrating B7-H3 expression in C4-2 cells. FIG. 22C is a graph illustrating B7-H3 expression in LN-CaP cells. FIG. 22D is a graph illustrating B7-H3 expression in enzalutamide resistant LNCap cells. FIG. 22E is a graph illustrating B7-H3 expression in PC3 cells. FIG. 22F is a graph illustrating B7-H3 expression in 22RV1 cells.

FIGS. 23A-23L illustrate how camB7-H3 TriKE induces activity against prostate cancer cells over a broader dynamic range than previous scFv version. FIG. 23A is a graph illustrating percent CD107a+ NK cells in PBMCNK alone in the presence of 0.3 nM TriKE. FIG. 23B is a graph illustrating percent CD107a+ NK cells in PBMCNK alone in the presence of 3 nM TriKE. FIG. 23C is a graph illustrating percent CD107a+ NK cells in PBMCNK alone in the presence of 30 nM TriKE. FIG. 23D is a graph illustrating percent CD107a+ NK cells in PBMCNK and CA-2 cells in the presence of 0.3 nM TriKE. FIG. 23E is a graph illustrating percent CD107a+ NK cells in PBMCNK and CA-2 cells in the presence of 3 nM TriKE. FIG. 23F is a graph illustrating percent CD107a+ NK cells in PBMCNK and CA-2 cells in the presence of 30 nM TriKE. FIG. 23G is a graph illustrating percent IFNγ+ NK cells in PBMCNK alone in the presence of 0.3 nM TriKE.

FIG. 23H is a graph illustrating percent IFNγ+ NK cells in PBMCNK alone in the presence of 3 nM TriKE. FIG. 23I is a graph illustrating percent IFNγ+ NK cells in PBMCNK alone in the presence of 30 nM TriKE. FIG. 23J is a graph illustrating percent IFNγ+ NK cells in PBMCNK and CA-2 cells in the presence of 0.3 nM TriKE. FIG. 23K is a graph illustrating percent IFNγ+ NK cells in PBMCNK and CA-2 cells in the presence of 3 nM TriKE. FIG. 23L is a graph illustrating percent IFNγ+ NK cells in PBMCNK and CA-2 cells in the presence of 30 nM TriKE.

FIGS. 24A-24F illustrate that B7-H3 TriKE enhances NK cell function against lung tumor targets. FIG. 24A is a graph showing pooled analysis of proportion of NK cells from healthy donors expressing CD107a+ against A549(N=6) tumor targets. FIG. 24B is a graph showing pooled analysis of proportion of NK cells from healthy donors expressing IFNγ against A549(N=6) tumor targets. FIG. 24C is a graph showing pooled analysis of proportion of NK cells from healthy donors expressing CD107a+ against NCI-H322 (N=5) tumor targets. FIG. 24D is a graph showing pooled analysis of proportion of NK cells from healthy donors expressing IFNγ against NCI-H322 (N=5) tumor targets. FIG. 24E is a graph showing pooled proportion of NK cells from healthy donors (black bars) or lung cancer patients (white bars) expressing CD107a+ against NCI-H460 (N=7). FIG. 24F is a graph showing pooled proportion of NK cells from healthy donors (black bars) or lung cancer patients (white bars) expressing IFNγ against NCI-H460 (N=7). ** p<0.01, *** p<0.001, and **** p<0.0001.

FIGS. 25A-25B illustrate the assessment of NK cell activity against HNSCC without treatment. FIG. 25A is a graph illustrating percent CD107a expression (as a marker for degranulation). FIG. 25B is a graph illustrating percent intracellular IFN-γ production.

FIGS. 26A-26B illustrate the assessment of B7-H3 expression on tumor and immune cells. FIG. 26A is a graph illustrating 5 HNSCC cell lines B7-H3 expression and binding affinity with B7-H3 single domain via flow cytometry. FIG. 26B is a graph illustrating PBMCs from a healthy donor B7-H3 expression by flow cytometry.

FIGS. 27A-27D illustrate the functional validation of B7-H3 TriKE. FIG. 27A is a graph illustrating CD107a expression in PBMCs from healthy donors incubated with Cal27 trio. FIG. 27B is a graph illustrating intracellular IFN-γ production in PBMCs from healthy donors incubated with Cal27 trio. FIG. 27C is a graph illustrating CD107a expression in PBMCs from healthy donors incubated with Cal33 trio. FIG. 27D is a graph illustrating intracellular IFN-γ production in PBMCs from healthy donors incubated with Cal33 trio.

FIGS. 28A-28F illustrate real-time imaging assays. FIG. 28A is a graph showing Nuclight red-labeled Cal27 survival after incubation with NK cells at an E:T of 5:1 in different conditions: no treatment or 3 nM IL-15, B7-H3 SD and B7-H3 TriKE for 48 hours in an IncuCyte Zoom imager. FIG. 28B shows photographs illustrating spheroids of Nuclight red-labeled Cal27. FIG. 28C is a graph illustrating the size of the spheroid in FIG. 28B. FIG. 28D is a graph showing Nuclight red-labeled Cal33 survival after incubation with NK cells at an E:T of 5:1 in different conditions: no treatment or 3 nM IL-15, B7-H3 SD and B7-H3 TriKE for 48 hours in an IncuCyte Zoom imager. FIG. 28E shows photographs illustrating spheroids of Nuclight red-labeled Cal33. FIG. 28F is a graph illustrating the size of the spheroid in FIG. 28E.

FIGS. 29A-29B show that various doses of cam1615B7-H3 TriKE enhance NK cell function against ovarian tumor targets. FIG. 29A is a graph illustrating pooled proportion of NK cells from healthy donors expressing CD107a+ (degranulation) against OVCAR8 and MA148 targets. FIG. 29B is a graph illustrating pooled proportion of NK cells from healthy donors expressing IFNγ cytokine production against OVCAR8 and MA148 targets.

FIGS. 30A-30I show that cam1615B7-H3 TriKE enhances NK cell function against ovarian tumor targets. Healthy donor or ovarian cancer PBMC cells were incubated with ovarian tumor targets at 2:1 E:T ratio for 4 hrs with 30 nM TriKE or control condition. FIG. 30A is a graph illustrating pooled proportion of NK cells from healthy donors expressing CD107a+ (degranulation) against OVCAR8 targets (N=8). FIG. 30B is a graph illustrating pooled proportion of NK cells from healthy donors expressing IFNγ cytokine production against OVCAR8 targets (N=8). FIG. 30C is a graph illustrating pooled proportion of NK cells from healthy donors expressing CD107a+ (degranulation) against OVCAR3 targets (N=4). FIG. 30D is a graph illustrating pooled proportion of NK cells from healthy donors expressing IFNγ cytokine production against OVCAR3 targets (N=4). FIG. 30E is a graph illustrating pooled proportion of NK cells from healthy donors expressing CD107a+ (degranulation) against OVCAR5 targets (N=7). FIG. 30F is a graph illustrating pooled proportion of NK cells from healthy donors expressing IFNγ cytokine production against OVCAR5 targets (N=7). FIG. 30G is a graph illustrating pooled analysis of proportion of NK cells from healthy donors (black bars) or ovarian cancer patients (white bars) expressing CD107a+ (degranulation) against MA-148 targets (N=9). FIG. 30H is a graph illustrating pooled analysis of proportion of NK cells from healthy donors (black bars) or ovarian cancer patients (white bars) expressing IFNγ against MA-148 targets (N=6). FIG. 30I is a graph illustrating that tumor killing was evaluated using IncuCyte imaging assay. NuclightRed expressing OVCAR8 targets were incubated with enriched healthy donor NK over 48 hrs, with Caspase 3/7 viability dye. The percentage of Live (Nuclight Red+Caspase 3/7−) tumor cells was quantified over a 48-h period and normalized to tumor alone. Readings were obtained every 15 min (representative of four separate experiments). * p<0.05, ** p<0.01, *** p<0.001, and **** p<0.0001.

FIG. 31 illustrates high dimensional analysis of cam1615B7-H3 activated NK cells. Concatenated analysis of three donors PBMCs incubated alone, with 30 nM cam1615B7-H3 TriKE, with OVCAR8 tumor (2:1 E:T), or with 30 nM cam1615B7-H3 TriKE and OVCAR8 tumor. Data was visualized in viSNE (Cytobank) and gated on CD45+CD56+CD3-cells.

FIGS. 32A-32F illustrate that cam1615B7-H3 TriKE is efficacious in subduing ovarian tumor progression in vivo. FIG. 32A is a diagram showing xenogeneic ovarian cancer MA-148-Luc model in female NSG mice (N=5 per treatment group). FIG. 32B is a graph showing bioluminescent imaging indicative of tumor progression, measured as Total flux radiance (p/s), over three weeks in the MA148 mouse model treated with enriched NK cells and noted treatments. FIG. 32C is a graph illustrating bioluminescent imaging results on day 21 data point. FIG. 32D is a photograph illustrating total flux radiance (p/s) on day 21. FIG. 32E is a scatter plot of CD56+CD3− NK cell numbers from peritoneal lavages for the rhIL-15 and cam1615B7-H3 TriKE treatment groups at D21. FIG. 32F is a scatter plot of CD16 media fluorescence intensity on NK cells from peritoneal lavages for the rhIL-15 and cam1615B7-H3 TriKE treatment groups at day 21. * p<0.05, ** p<0.01.

### DETAILED DESCRIPTION OF THE INVENTION

The present invention is based on the development of B7-H3 targeting fusion proteins, and specifically B7-H3 targeting tri-specific killer engager molecules (TriKEs) and methods of use thereof.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides an isolated nucleic acid sequence as set forth in SEQ ID NO:13 or 14 or a sequence having 90% identity thereto.

As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be employed for introduction into, i.e., transfection of, cells, in particular, in the form of RNA which can be prepared by in vitro transcription from a DNA template. The RNA can moreover be modified before application by stabilizing sequences, capping, and polyadenylation.

As used herein “amplified DNA” or “PCR product” refers to an amplified fragment of DNA of defined size. Various techniques are available and well known in the art to detect PCR products. PCR product detection methods include, but are not restricted to, gel electrophoresis using agarose or polyacrylamide gel and adding ethidium bromide staining (a DNA intercalant), labeled probes (radioactive or non-radioactive labels, southern blotting), labeled deoxyribonucleotides (for the direct incorporation of radioactive or non-radioactive labels) or silver staining for the direct visualization of the amplified PCR products; restriction endonuclease digestion, that relies agarose or polyacrylamide gel or High-performance liquid chromatography (HPLC); dot blots, using the hybridization of the amplified DNA on specific labeled probes (radioactive or non-radioactive labels); high-pressure liquid chromatography using ultraviolet detection; electro-chemiluminescence coupled with voltage-initiated chemical reaction/photon detection; and direct sequencing using radioactive or fluorescently labeled deoxyribonucleotides for the determination of the precise order of nucleotides with a DNA fragment of interest, oligo ligation assay (OLA), PCR, qPCR, DNA sequencing, fluorescence, gel electrophoresis, magnetic beads, allele specific primer extension (ASPE) and/or direct hybridization.

Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; U.S. Pub. 2010/0285578; and U.S. Pub. 2002/0190663. Examples of nucleic acid analysis include, but are not limited to, sequencing and DNA-protein interaction. Sequencing may be by any method known in the art. DNA sequencing techniques include classic dideoxy sequencing reactions (Sanger method) using labeled terminators or primers and gel separation in slab or capillary, and next generation sequencing methods such as sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence (e.g., SEQ ID NO:13 or 14) aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 85%, at least 90%, at least 95%, at least 96%, at least 97%, at least 98%, at least 99%, at least 99.5%, or at least 99.9%. In general, an exact match indicates 100% identity over the length of the reference sequence (e.g., SEQ ID NO:13 or 14). Preferably, sequences that are not 100% identical to sequences provided herein retain the function of the original sequence (e.g., ability to bind B7-H3 or CD16).

Polypeptides and polynucleotides that are about 85, 86, 87, 88, 89, 90, 91, 92, 93, 94, 95, 96, 97, 98, 99 99.5% or more identical to polypeptides and polynucleotides described herein are embodied within the disclosure. For example, a polypeptide can have 80%, 85%, 90%, 91%, 92%, 93%, 94%, 95%, 96%, 97%, 98%, 99% or more identity to SEQ ID NO:13 or 14.

Variants of the disclosed sequences also include peptides, or full-length protein, that contain substitutions, deletions, or insertions into the protein backbone, that would still leave at least about 70% homology to the original protein over the corresponding portion. A yet greater degree of departure from homology is allowed if like-amino acids, i.e., conservative amino acid substitutions, do not count as a change in the sequence. Examples of conservative substitutions involve amino acids that have the same or similar properties. Illustrative amino acid conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

In another embodiment, the invention provides a protein encoded by a nucleic acid sequence as set forth in SEQ ID NO:13 or 14 or a sequence having 90% identity thereto.

The terms “peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

In one aspect, the amino acid sequence is selected from SEQ ID NO:6 or 7.

The nucleic acid sequences provided herein can encode for example a light chain or a heavy chain of an antibody, conferring to the encoded polypeptide a binding domain or targeting domain to a specific target. Such a polypeptide can be referred to as a targeting peptide.

The term “antibody” generally refers to immunoglobulin molecules and immunologically active portions of immunoglobulin molecules, i.e., molecules that contain an antigen binding site that immunospecifically binds an antigen. “Native antibodies” and “intact immunoglobulins”, or the like, are usually heterotetrameric glycoproteins of about 150,000 daltons, composed of two identical light (L) chains and two identical heavy (H) chains. The light chains from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known.

In a typical antibody molecule, each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains. Each variable region includes three segments called complementarity-determining regions (CDRs) or hypervariable regions and a more highly conserved portions of variable domains are called the framework region (FR). The variable domains of heavy and light chains each includes four FR regions, largely adopting a 3-sheet configuration, connected by three CDRs, which form loops connecting, and in some cases forming part of the 3-sheet structure. The CDRs in each chain are held together in close proximity by the FRs and, with the CDRs from the other chain, contribute to the formation of the antigen-binding domain or targeting domain of antibodies (see Kabat et al., NIH Publ. No. 91-3242, Vol. I, pages 647-669 [1991]). The constant domains are not involved directly in binding an antibody to an antigen, but exhibit various effector functions, such as participation of the antibody in antibody dependent cellular cytotoxicity.

Antibodies can be cleaved experimentally with the proteolytic enzyme papain, which causes each of the heavy chains to break, producing three separate antibody fragments. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding but is important in later processes involved in ridding the body of the antigen. As used herein, “antibody fragments” include a portion of an intact antibody, preferably the antigen binding or variable region of the intact antibody. Examples of antibody fragments include Fab, Fab′ and F(ab′)2, Fc fragments or Fc-fusion products, single-chain Fvs (scFv), disulfide-linked Fvs (sdfv) and fragments including either a VL or VH domain; diabodies, tribodies and the like (Zapata et al. Protein Eng. 8(10):1057-1062 [1995]).

The Fab fragment contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′) 2 antibody fragments originally were produced as pairs of Fab′ fragments which have hinge cysteines between them. Other chemical couplings of antibody fragments are also known.

The Fc region of an antibody is the tail region of an antibody that interacts with cell surface receptors and some proteins of the complement system. This property allows antibodies to activate the immune system. In IgG, IgA and IgD antibody isotypes, the Fc region is composed of two identical protein fragments, derived from the second and third constant domains of the antibody's two heavy chains; IgM and IgE Fc regions contain three heavy chain constant domains (CH domains 2-4) in each polypeptide chain. The Fc regions of IgGs bear a highly conserved N-glycosylation site. Glycosylation of the Fc fragment is essential for Fc receptor-mediated activity. The N-glycans attached to this site are predominantly core-fucosylated diantennary structures of the complex type. In addition, small amounts of these N-glycans also bear bisecting GlcNAc and α-2,6 linked sialic acid residues.

Fc-Fusion proteins (also known as Fc chimeric fusion protein, Fc-Ig, Ig-based Chimeric Fusion protein and Fc-tag protein) are composed of the Fc domain of IgG genetically linked to a peptide or protein of interest. Fc-Fusion proteins have become valuable reagents for in vivo and in vitro research. The Fc-fused binding partner can range from a single peptide, a ligand that activates upon binding with a cell surface receptor, signaling molecules, the extracellular domain of a receptor that is activated upon dimerization or as a bait protein that is used to identify binding partners in a protein microarray. One of the most valuable features of the Fc domain in vivo, is it can dramatically prolong the plasma half-life of the protein of interest, which for bio-therapeutic drugs, results in an improved therapeutic efficacy; an attribute that has made Fc-Fusion proteins attractive bio-therapeutic agents. The Fc fusion protein may be part of a pharmaceutical composition including an Fc fusion protein and a pharmaceutically acceptable carrier excipients or carrier. Pharmaceutically acceptable carriers, excipients or stabilizers are well known in the art (Remington's Pharmaceutical Sciences, 16th edition, Osol, A. Ed. (1980)). Acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 [1985]). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′2 fragments (Carter et al., Bio/Technology 10:163-167 [1992]). According to another approach, F(ab′) 2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

In various aspects, the nucleic acid sequences provided herein encode a light chain and a heavy chain that bind specifically to a B3-H7 protein.

B7 Homolog 3 (B7-H3) also known as cluster of differentiation 276 (CD276) is a human protein encoded by the CD276 gene. The B7-H3 protein is a 316 amino acid-long type I transmembrane protein existing in two isoforms determined by its extracellular domain. In mice, the extracellular domain consists of a single pair of immunoglobulin variable (IgV)-like and immunoglobulin constant (IgC)-like domains, whereas in humans it consists of one pair (2Ig-B7-H3) or two identical pairs (4Ig-B7-H3) due to exon duplication. B7-H3 mRNA is expressed in most normal tissues. In contrast, B7-H3 protein has a very limited expression on normal tissues because of its post-transcriptional regulation by microRNAs. However, B7-H3 protein is expressed at high frequency on many different cancer types (60% of all cancers).

In non-malignant tissues, B7-H3 has a predominantly inhibitory role in adaptive immunity, suppressing T cell activation and proliferation. In malignant tissues, B7-H3 is an immune checkpoint molecule that inhibits tumor antigen-specific immune responses. B7-H3 also possesses non-immunological pro-tumorigenic functions such as promoting migration, invasion, angiogenesis, chemoresistance, epithelial-to-mesenchymal transition, and affecting tumor cell metabolism. Due to its selective expression on solid tumors and its pro-tumorigenic function, B7-H3 is the target of several anti-cancer agents including enoblituzumab, omburtamab, MGD009, MGC018, DS-7300a, and CAR-T cells.

As used herein, the term “B7-H3 targeting peptide” or “B7-H3 targeting protein” is meant to refer to any peptide or polypeptide (including protein and fusion protein) that can specifically bind to B7-H3. The B7-H3 targeting peptide can be an antibody, an antibody fragment, and the like, having specific binding to one or more target polypeptide, including B7-H3. In some aspects, the polypeptide encodes the light chain and the heavy chain of a B7-H3 targeting peptide. In one aspect, the nucleic acid sequence of SEQ ID NO:13 can encode the light chain of a B7-H3 targeting peptide, having the amino acid sequence as set forth in SEQ ID NO:6. In another aspect, the nucleic acid sequence of SEQ ID NO:14 can encode the heavy chain of a B7-H3 targeting peptide, having the amino acid sequence as set forth in SEQ ID NO:8.

In an additional embodiment, the invention provides a fusion protein including the amino acid sequence set forth in SEQ ID NO:6 and 7, operably linked to each other in either orientation.

The terms “fusion molecule” and “fusion protein” are used interchangeably and are meant to refer to a biologically active polypeptide, with or without a further effector molecule, usually a protein or peptide sequence covalently linked (i.e., fused) by recombinant, chemical or other suitable method. If desired, the fusion molecule can be used at one or several sites through a peptide linker sequence. Alternatively, the peptide linker may be used to assist in construction of the fusion molecule. Specifically, preferred fusion molecules are fusion proteins. Generally fusion molecule also can include conjugate molecules.

By “operably linked” to one another, it is meant that there is a direct or indirect covalent linking between the peptides composing the fusion protein. Thus, two domains that are operably linked may be directly covalently coupled to one another. Conversely, the two operably linked domains may be connected by mutual covalent linking to an intervening moiety (e.g., and flanking sequence). Two domains may be considered operably linked if, for example, they are separated by the third domain, with or without one or more intervening flanking sequences.

Methods for attaching two individual elements usually require the use of a linker. The term “linker” as used herein refers any bond, small molecule, or other vehicle which allows the substrate and the active agent to be targeted to the same area, tissue, or cell, for example by physically linking the individual portions of the conjugate. A linker can be any chemical moiety that is capable of linking a compound, usually a drug, to a cell-binding agent in a stable, covalent manner.

The fusion proteins provided herein can for example include the amino acid sequences set forth in SEQ ID NOs:6 and 7, operably linked to each other in either orientation. For example, the fusion protein can include the amino acid sequence set forth in SEQ ID NO:6 at a C-terminal of the fusion protein and the amino acid sequence set forth in SEQ ID NO:7 at a N-terminal of the fusion protein; or the fusion protein can include the amino acid sequence set forth in SEQ ID NO:6 at a N-terminal of the fusion protein and the amino acid sequence set forth in SEQ ID NO:7 at a C-terminal of the fusion protein. The orientation of the amino acid sequences in the fusion protein do not alter the binding-specificity of the fusion protein to its target (i.e., B7-H3 targeting fusion protein).

The light chain and the heavy chain of the B7-H3 targeting peptide can be operably linked to one another in either orientation without affecting the binding specificity or sensitivity of the targeting peptide. In one aspect, the protein includes SEQ ID NO:6 and 7, in direct linkage between the C-terminus of SEQ ID NO:6 and the N-terminus of SEQ ID NO:7. In another aspect, the protein includes SEQ ID NO:7 and 6, in direct linkage between the C-terminus of SEQ ID NO:7 and the N-terminus of SEQ ID NO:6.

The fusion protein provided herein can include additional protein domain, such as additional targeting domain to provide the fusion protein with specific binding to one or more target polypeptide. For example, the fusion protein can be a tri-specific killer engager (TriKE) molecule including the B7-H3 targeting peptide as the targeting domain.

NK cells are cytotoxic lymphocytes of the innate immune system capable of immune surveillance. Like cytotoxic T cells, NK cells deliver a store of membrane penetrating and apoptosis-inducing granzyme and perforin granules. Unlike T cells, NK cells do not require antigen priming and recognize targets by engaging activating receptors in the absence of MHC recognition. NK cells express CD16, an activation receptor that binds to the Fc portion of IgG antibodies and is involved in antibody-dependent cell-mediated cytotoxicity (ADCC). NK cells are regulated by IL-15, which can induce increased antigen-dependent cytotoxicity, lymphokine-activated killer activity, and/or mediate interferon (IFN), tumor-necrosis factor (TNF) and/or granulocyte-macrophage colony-stimulating factor (GM-CSF) responses. All of these IL-15-activated functions contribute to improved cancer defense.

Therapeutically, adoptive transfer of NK cells can, for example, induce remission in patients with refractory acute myeloid leukemia (AML) when combined with lymphodepleting chemotherapy and IL-2 to stimulate survival and in vivo expansion of NK cells. This therapy can be limited by lack of antigen specificity and IL-2-mediated induction of regulatory T (Treg) cells that suppress NK cell proliferation and function. Generating a reagent that drives NK cell antigen specificity, expansion, and/or persistence, while bypassing the negative effects of Treg inhibition, can enhance NK-cell-based immunotherapies.

Tri-specific killer engager molecule are targeting fusion protein including two domains capable of driving NK-cell-mediated killing of tumor cells (e.g., CD33+ tumor cells and/or EpCAM+ tumor cells) and an intramolecular NK activating domain capable of generating an NK cell self-sustaining signal can drive NK cell proliferation and/or enhance NK-cell-driven cytotoxicity against, for example, HL-60 targets, cancer cells, or cancer cell-derived cell lines.

NK cells are responsive to a variety of cytokines including, for example, IL-15, which is involved in NK cell homeostasis, proliferation, survival, activation, and/or development. IL-15 and IL-2 share several signaling components, including the IL-2/IL-15Rβ (CD122) and the common gamma chain (CD132). Unlike IL-2, IL-15 does not stimulate Tregs, allowing for NK cell activation while bypassing Treg inhibition of the immune response. Besides promoting NK cell homeostasis and proliferation, IL-15 can rescue NK cell functional defects that can occur in the post-transplant setting. IL-15 also can stimulate CD8+ T cell function, further enhancing its immunotherapeutic potential. In addition, based on pre-clinical studies, toxicity profiles of IL-15 may be more favorable than IL-2 at low doses. IL-15 plays a role in NK cell development homeostasis, proliferation, survival, and activation. IL-15 and IL-2 share several signaling components including the IL-2/IL-15Rβ (CD122) and the common gamma chain (CD132). IL-15 also activates NK cells and can restore functional defects in engrafting NK cells after hematopoietic stem cell transplantation (HSCT).

The fusion protein provided herein can be a TriKE molecule including one or more NK cell engager domains (e.g., CD16, CD16+CD2, CD16+DNAM, CD16+NKp46), one or more targeting domains (that target, e.g., a tumor cell or virally-infected cell, such as the B7-H3 targeting peptide described herein), and one or more cytokine NK activating domains (e.g., IL-15, IL-12, IL-18, IL-21, or other NK cell enhancing cytokine, chemokine, and/or activating molecule), with each domain operably linked to the other domains.

For example, the fusion protein described herein can be a TriKE molecule including a CD16 NK cell engager domain, such as the CD16 domain having the amino acid sequence set forth in SEQ ID NO:2 or 19; a B7-H3 targeting fusion protein domain, such as the B7-H3 fusion protein having the amino acid sequences set forth in SEQ ID NOs:6 and 7; and a IL-15 cytokine NK activating domain, such as the IL-15 having the amino acid sequence set forth in SEQ ID NO:4, 17 or 18.

The different protein domains of the TriKE molecules can be in operable linkage with one another. For example, linkers can be used to covalently attached the protein domains of the TriKE molecule to one another.

The elements of a fusion protein can be in assembled operable linkage with one another using one or more linkers. Linkers can be susceptible to or be substantially resistant to acid-induced cleavage, light-induced cleavage, peptidase-induced cleavage, esterase-induced cleavage and disulfide bond cleavage at conditions under which the compound or the antibody remains active. Linkers are classified upon their chemical motifs, well known in the art, including disulfide groups, hydrazine or peptides (cleavable), or thioester groups (non-cleavable). Linkers also include charged linkers, and hydrophilic forms thereof as known in the art.

Suitable linker for the fusion of two or more protein or protein domains can include natural linkers, and empirical linkers. Natural linkers are derived from multi-domain proteins, which are naturally present between protein domains. Natural linkers can have several properties depending or their such as length, hydrophobicity, amino acid residues, and secondary structure, which can impact the fusion protein in different way.

The studies of linkers in natural multi-domain proteins have led to the generation of many empirical linkers with various sequences and conformations for the construction of recombinant fusion proteins. Empirical linkers can be classified in three types: flexible linkers, rigid linkers, and cleavable linkers. Flexible linkers can provide a certain degree of movement or interaction at the joined domains. They are generally composed of small, non-polar (e.g., Gly) or polar (e.g., Ser or Thr) amino acids, which provides flexibility, and allows for mobility of the connecting functional domains. Rigid linkers can successfully keep a fixed distance between the domains to maintain their independent functions, which can provide efficient separation of the protein domains or sufficient reduction of their interference with each other. Cleavable linkers can allow the release of functional domains in vivo. By taking advantage of unique in vivo processes, they can be cleaved under specific conditions such as the presence of reducing reagents or proteases. This type of linker can reduce steric hindrance, improve bioactivity, or achieve independent actions/metabolism of individual domains of recombinant fusion proteins after linker cleavage.

Non limiting examples of linker include linkers having the amino acid sequences set forth in SEQ ID NOs: 3, 5, 10, 12, 15 and 16.

In one aspect, SEQ ID NO:2 or 19, and 4, 17 or 18 are linked by SEQ ID NO:3 or SEQ ID NO:15. In another aspect, SEQ ID NO:4, 17 or 18 and 6 or 7 are linked by SEQ ID NO:5 or SEQ ID NO:16. In other aspects, SEQ ID NO:6 and 7 are in operable linkage in either orientation.

In a further embodiment, the invention provides a fusion protein including the sequence set forth in SEQ ID NO:1 and sequences having 90% or greater identity to SEQ ID NO:1.

In one embodiment, the invention provides a fusion protein including in operably linkage, SEQ ID NO:2 or 19; 4, 17, or 18; 6 and 7, or 7 and 6.

The fusion protein described herein can include a wild-type (wt) IL-15 or mutant IL-15 cytokine NK activating domain. Mutant IL-15 can for example include IL-15 including a substitution of the N72 amino acid. Non-limiting examples of N72 substitutions include N72A and N72D mutations.

In some aspects, SEQ ID NO:4 has an N72 substitution. In various aspects, the N72 mutation is N72A or N72D and the protein is set forth in SEQ ID NO:17 or 18, respectively.

In yet another embodiment, the invention provides a fusion protein including SEQ ID NO:19, SEQ ID NO:17 or 18 and SEQ ID NO:6 and 7 in either orientation.

In one aspect, SEQ ID NO:19 is operably linked to SEQ ID NO:17 or 18 by a linker of SEQ ID NO:3 or 15. In another aspect, SEQ ID NO:17 or 18 is operably linked to SEQ 6 and 7, in either orientation by a linker of SEQ ID NO:5 or 16.

The fusion protein can include in operable linkage a camelid or a human CD16 NK cell engager domain (SEQ ID NO:2 or 19, respectively), a wt or a mutant IL-15 cytokine NK activating domain (SEQ ID NO:4, 17 or 18), and a light chain and a heavy chain of an of a B7-H3 targeting peptide (SEQ ID NO:6 and 7, respectively). The CD16 NK cell engager domain can be linked to IL-15 cytokine NK activating domain by a linker having an amino acid sequence set forth in SEQ ID NO:3 or 15. The IL-15 cytokine NK activating domain can be linked to the B7-H3 targeting peptide by a linker having an amino acid sequence set forth in SEQ ID NO:5 or 16. The IL-15 cytokine NK activating domain can be linked to the heavy chain of the B7-H3 targeting peptide (linked to the light chain), or to the light chain of the B7-H3 targeting peptide (linked to the heavy chain).

For example, the fusion protein can include, in operable linkage, from an N-terminus to a C-terminus, SEQ ID NOs:2, 4, 6 and 7; SEQ ID NOs:2, 4, 7 and 6; SEQ ID NOs:19, 17, 6 and 7; SEQ ID NOs:19, 17, 7 and 6; SEQ ID NOs:19, 18, 6 and 7; or SEQ ID NOs:19, 18, 7 and 6.

Specifically, the fusion protein can include, in operable linkage, from a N-terminus to a C-terminus, SEQ ID NOs:2, 3, 4, 5, 6 and 7; SEQ ID NOs:2, 3, 4, 16, 6 and 7; SEQ ID NOs:2, 15, 4, 5, 6 and 7; SEQ ID NOs:2, 15, 4, 16, 6 and 7; SEQ ID NOs:2, 3, 4, 5, 7 and 6; SEQ ID NOs:2, 3, 4, 16, 7 and 6; SEQ ID NOs:2, 15, 4, 5, 7 and 6; or SEQ ID NOs:2, 15, 4, 16, 7 and 6.

In other aspects, the fusion protein can include, in operable linkage, from a N-terminus to a C-terminus, SEQ ID NOs:19, 3, 17, 5, 6 and 7; SEQ ID NOs:19, 3, 17, 16, 6 and 7; SEQ ID NOs:19, 15, 17, 5, 6 and 7; SEQ ID NOs:19, 15, 17, 16, 6 and 7; SEQ ID NOs:19, 3, 17, 5, 7 and 6; SEQ ID NOs:19, 3, 17, 16, 7 and 6; SEQ ID NOs:19, 15, 17, 5, 7 and 6; SEQ ID NOs:19, 15, 17, 16, 7 and 6; SEQ ID NOs:19, 3, 18, 5, 6 and 7; SEQ ID NOs:19, 3, 18, 16, 6 and 7; SEQ ID NOs:19, 15, 18, 5, 6 and 7; SEQ ID NOs:19, 15, 18, 16, 6 and 7; SEQ ID NOs:19, 3, 18, 5, 7 and 6; SEQ ID NOs:19, 3, 18, 16, 7 and 6; SEQ ID NOs:19, 15, 18, 5, 7 and 6; or SEQ ID NOs:19, 15, 18, 16, 7 and 6.

In some aspects, the fusion protein further includes a half-life extending (HLE) molecule.

The circulatory half-life of targeting proteins such as IgG immunoglobulins can be regulated by the affinity of the Fc region for the neonatal Fc receptor (FcRn). The second general category of effector functions include those that operate after an immunoglobulin binds an antigen. In the case of IgG, these functions involve the participation of the complement cascade or Fe gamma receptor (FcγR)-bearing cells. Binding of the Fc region to an FcγR causes certain immune effects, for example, endocytosis of immune complexes, engulfment and destruction of immunoglobulin-coated particles or microorganisms (also called antibody-dependent phagocytosis, or ADCP), clearance of immune complexes, lysis of immunoglobulin-coated target cells by killer cells (called antibody-dependent cell-mediated cytotoxicity, or ADCC), release of inflammatory mediators, regulation of immune system cell activation, and regulation of immunoglobulin production. Certain engineered binding polypeptides (e.g., antibody variants (e.g., scFvs) or antibody fragments (e.g., Fab fragments)), while benefiting from their smaller molecular size and/or monovalency, also suffer several disadvantages attributable to the absence of a functional Fc region. For example, Fab fragments have short half-lives in vivo because they lack the Fc region that is required for FcRn binding and are rapidly filtered out of the blood by the kidneys owing to their small size.

Engineered targeting polypeptides, such as the fusion proteins described herein, can exhibit decreased binding to FcRn when compared to native binding polypeptides and, therefore, have decreased half-life in vivo. Fc variants with improved affinity for FcRn can have longer serum half-lives, and such molecules have useful applications in methods of treating mammals where long half-life of the administered polypeptide is desired, e.g., to treat a chronic disease or disorder. In contrast, Fc variants with decreased FcRn binding affinity have shorter half-lives, and such molecules are also useful, for example, for administration to a mammal where a shortened circulation time may be advantageous, e.g., for in vivo diagnostic imaging or in situations where the starting polypeptide has toxic side effects when present in the circulation for prolonged periods.

The fusion proteins described herein can include a half-life extending (HLE) molecule to extend their half-life in vivo upon administration to a subject.

As used herein, the term “half-life” refers to a biological half-life of a particular targeting polypeptide in vivo. Half-life may be represented by the time required for half the quantity administered to a subject to be cleared from the circulation and/or other tissues in the animal. When a clearance curve of a targeting polypeptide is constructed as a function of time, the curve is usually biphasic with a rapid α-phase and longer β-phase. The α-phase typically represents an equilibration of the administered targeting polypeptide between the intra- and extra-vascular space and is, in part, determined by the size of the polypeptide. The β-phase typically represents the catabolism of the targeting polypeptide in the intravascular space. Therefore, the term half-life as used herein preferably refers to the half-life of the targeting polypeptide in the β-phase. The typical β phase half-life of a human antibody in humans is 21 days.

An increased half-life is generally useful in in vivo applications of immunoglobulins, especially antibodies and most especially antibody fragments of small size. Approaches described in the art to achieve such effect comprise the fusion of the small bispecific antibody construct to larger proteins, which preferably do not interfere with the therapeutic effect of the protein construct. Examples for such further developments of bispecific T cell engagers are described in US 2017/0218078A1, which provides half-life extending formats (HLE formats) of bispecific T cell engaging molecules comprising a first domain binding to a target cell surface antigen, a second domain binding to an extracellular epitope of the human and/or the Macaca CD3ε chain and a third domain, which is the specific Fc modality (the HLE molecule).

As used herein, the terms “half-life extending molecule”, “HLE sequence” and the like are meant to refer to any molecule, such as a protein or polypeptide that can be linked or fused to a polypeptide of interest to increase or extend its half-life in vivo. Specifically, an HLE sequence generally includes a Fc region or scFc region of an immunoglobulin.

As used herein, the term “Fc region” refers to the portion of a native immunoglobulin formed by the respective Fc domains (or Fc moieties) of its two heavy chains. A native Fc region is homodimeric. In contrast, the term “genetically-fused Fc region” or “single-chain Fc region” (scFc region), as used herein, refers to a synthetic Fc region comprised of Fc domains (or Fc moieties) genetically linked within a single polypeptide chain (i.e., encoded in a single contiguous genetic sequence). Accordingly, a genetically fused Fc region (i.e., a scFc region) is monomeric.

The term “Fc domain” refers to the portion of a single immunoglobulin heavy chain beginning in the hinge region just upstream of the papain cleavage site (i.e., residue 216 in IgG, taking the first residue of heavy chain constant region to be 114) and ending at the C-terminus of the antibody. Accordingly, a complete Fc domain comprises at least a hinge domain, a CH2 domain, and a CH3 domain.

The scFc region described herein includes at least two Fc domain which are genetically fused via a linker polypeptide (e.g., an Fc connecting peptide) interposed between said Fc moieties. The scFc region can include two identical Fc moieties or can include two non-identical Fc moieties.

Non-limiting examples of Fc domain that can be used for the preparation of a HLE molecule (alone or in combination with another Fc domain through a linker polypeptide) that can be incorporated in any of the fusion proteins described herein include any of the polypeptides having an amino acid including any one of SEQ ID NOs:26-33.

Non-limiting examples of linker polypeptide that can be used for the preparation of a scFc region that can be used for the preparation of a HLE molecule include any of the polypeptides having an amino acid including any one of SEQ ID NOs:34-35.

The HLE molecules described herein can include a Fc domain having an amino acid including any one of SEQ ID NOs:26-33, or a scFc region including a first Fc domain having an amino acid comprising any one of SEQ ID NOs:26-33 fused to a second Fc domain having an amino acid comprising any one of SEQ ID NOs:26-33, through a linker having an amino acid including any one of SEQ ID NOs:34-35. For example, the HLE molecule can include any one of SEQ ID NOs:21-25.

In an additional embodiment, the invention provides an isolated nucleic acid sequence encoding any of the fusion proteins described herein.

The fusion proteins described herein, such as the TriKE fusion proteins including a CD16 NK cell engager domain, such as the CD16 domain having the amino acid sequence set forth in SEQ ID NO:2; a B7-H3 targeting fusion protein domain, such as the B7-H3 fusion protein having the amino acid sequences set forth in SEQ ID NOs:6 and 7; and a IL-15 cytokine NK activating domain, such as the IL-15 having the amino acid sequence set forth in SEQ ID NO:4, in operable linkage, and as set forth in SEQ ID NO:1 can be encoded by a nucleic acid sequence. In one aspect, the sequence is SEQ ID NO:8 or sequences having 90% or more sequence identity thereto.

In another embodiment, the invention provides a method of treating cancer in a subject comprising administering to the subject any of the fusion proteins described herein, thereby treating the cancer.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome. Such amount should be sufficient to treat cancer. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

The fusion proteins described herein can be formulated in pharmaceutical compositions comprising the fusion protein and a pharmaceutically acceptable carrier. By “pharmaceutically acceptable” it is meant the carrier, diluent or excipient must be compatible with the other ingredients of the formulation and not deleterious to the recipient thereof. Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

Pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

The methods described herein are directed to the treatment of cancer. The term “cancer” refers to a group of diseases characterized by abnormal and uncontrolled cell proliferation starting at one site (primary site) with the potential to invade and to spread to other sites (secondary sites, metastases) which differentiates cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and/or alcohol use, obesity, poor diet, lack of physical activity or any combination thereof. As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, brain, and head and neck cancers, melanoma, sarcoma, multiple myeloma, leukemia, lymphoma, and the like.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland's Cancer, Childhood; Sarcoma, Ewing's Family of Tumors; Sarcoma, Kaposi's; Sarcoma (Osteosarcoma) Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In one aspect, the cancer is selected from non-small lung cancer, cutaneous squamous cell carcinoma, pancreatic cancer, primary hepatocellular carcinoma, colorectal carcinoma, clear cell renal carcinoma or breast cancer.

In some aspects, administration of the fusion proteins described herein can be in combination with one or more additional therapeutic agents. The phrases “combination therapy”, “combined with” and the like refer to the use of more than one medication or treatment simultaneously to increase the response. The fusion proteins of the present invention and the pharmaceutical composition thereof might for example be used in combination with other drugs or treatment in use to treat cancer. Specifically, the administration of the fusion proteins to a subject can be in combination with a chemotherapeutic agent, surgery, radiotherapy, or a combination thereof. Such therapies can be administered prior to, simultaneously with, or following administration of the composition of the present invention.

The term “chemotherapeutic agent” as used herein refers to any therapeutic agent used to treat cancer. Examples of chemotherapeutic agents include, but are not limited to, Actinomycin, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, lrinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, Vinorelbine, panitumamab, Erbitux (cetuximab), matuzumab, IMC-IIF 8, TheraCIM hR3, denosumab, Avastin (bevacizumab), Humira (adalimumab), Herceptin (trastuzumab), Remicade (infliximab), rituximab, Synagis (palivizumab), Mylotarg (gemtuzumab oxogamicin), Sarclisa (isatuximab), Raptiva (efalizumab), Tysabri (natalizumab), Zenapax (dacliximab), NeutroSpec (Technetium (99mTc) fanolesomab), tocilizumab, ProstaScint (Indium-Ill labeled Capromab Pendetide), Bexxar (tositumomab), Zevalin (ibritumomab tiuxetan (IDEC-Y2B8) conjugated to yttrium 90), Xolair (omalizumab), MabThera (Rituximab), ReoPro (abciximab), MabCampath (alemtuzumab), Simulect (basiliximab), LeukoScan (sulesomab), CEA-Scan (arcitumomab), Verluma (nofetumomab), Panorex (Edrecolomab), alemtuzumab, CDP 870, natalizumab Gilotrif (afatinib), Lynparza (olaparib), Perjeta (pertuzumab), Otdivo (nivolumab), Bosulif (bosutinib), Cabometyx (cabozantinib), Ogivri (trastuzumab-dkst), Sutent (sunitinib malate), Adcetris (brentuximab vedotin), Alecensa (alectinib), Calquence (acalabrutinib), Yescarta (ciloleucel), Verzenio (abemaciclib), Keytruda (pembrolizumab), Aliqopa (copanlisib), Nerlynx (neratinib), Imfinzi (durvalumab), Darzalex (daratumumab), Tecentriq (atezolizumab), and Tarceva (erlotinib). Examples of immunotherapeutic agent include, but are not limited to, interleukins (11-2, 11-7, 11-12), cytokines (Interferons, G-CSF, imiquimod), chemokines (CCL3, CC126, CXCL7), immunomodulatory imide drugs (thalidomide and its analogues).

In one embodiment, the invention provides a pharmaceutical composition including a therapeutically effective amount of a fusion protein including the amino acid sequence of SEQ ID NO:1 or a sequence having 90% or greater identity to SEQ ID NO:1 and a pharmaceutically acceptable carrier.

In another embodiment, the invention provides a method of treating cancer in a subject including administering to the subject the pharmaceutical composition described herein.

Natural killer cells, also known as NK cells or large granular lymphocytes (LGL), are a type of cytotoxic lymphocyte critical to the innate immune system that belong to the rapidly expanding family of known innate lymphoid cells (ILC) and represent 5-20% of all circulating lymphocytes in humans. The role of NK cells is analogous to that of cytotoxic T cells in the vertebrate adaptive immune response. NK cells provide rapid responses to virus-infected cell and other intracellular pathogens acting at around 3 days after infection and respond to tumor formation. Typically, immune cells detect the major histocompatibility complex (MHC) presented on infected cell surfaces, triggering cytokine release, causing the death of the infected cell by lysis or apoptosis. NK cells are unique, however, as they have the ability to recognize and kill stressed cells in the absence of antibodies and MHC, allowing for a much faster immune reaction. They were named “natural killers” because of the notion that they do not require activation to kill cells that are missing “self” markers of MHC class 1. This role is especially important because harmful cells that are missing MHC I markers cannot be detected and destroyed by other immune cells, such as T lymphocyte cells.

In addition to natural killer cells being effectors of innate immunity, both activating and inhibitory NK cell receptors play important functional roles, including self-tolerance and the sustaining of NK cell activity. NK cells also play a role in the adaptive immune response: numerous experiments have demonstrated their ability to readily adjust to the immediate environment and formulate antigen-specific immunological memory, fundamental for responding to secondary infections with the same antigen. The role of NK cells in both the innate and adaptive immune responses is becoming increasingly important in research using NK cell activity as a potential cancer therapy.

In an additional embodiment, the invention provides a method of inducing natural killer (NK) cell activity against a cancer cell in a subject including administering to the subject a fusion protein including the sequence set forth in SEQ ID NO:1 and sequences having 90% or greater identity to SEQ ID NO:1, thereby inducing NK cell activity against a cancer cell in the subject.

In one aspect, inducing NK cell activity includes inducing NK cells degranulation, inducing NK cell production of interferon γ, increasing a number of tumor infiltrating NK cells in the subject, and/or inducing or increasing NK cell proliferation.

Natural killer cells or large granular lymphocytes (LGL) are a type of cytotoxic lymphocyte critical to the innate immune system that belong to the rapidly expanding family of known innate lymphoid cells (ILC) and represent 5-20% of all circulating lymphocytes in humans. They have different functions including: cytolytic granule mediated cell apoptosis, antibody-dependent cell-mediated cytotoxicity (ADCC) and cytokine-induced NK and cytotoxic T lymphocyte (CTL) activation.

NK cells are cytotoxic; small granules in their cytoplasm contain proteins such as perforin and proteases known as granzymes. Upon release in close proximity to a cell slated for killing, perforin forms pores in the cell membrane of the target cell, creating an aqueous channel through which the granzymes and associated molecules can enter, inducing either apoptosis or osmotic cell lysis. The distinction between apoptosis and cell lysis is important in immunology: lysing a virus-infected cell could potentially release the virions, whereas apoptosis leads to destruction of the virus inside. α-defensins, antimicrobial molecules, are also secreted by NK cells, and directly kill bacteria by disrupting their cell walls in a manner analogous to that of neutrophils.

Infected cells are routinely opsonized with antibodies for detection by immune cells. Antibodies that bind to antigens can be recognized by FcγRIII (CD16) receptors expressed on NK cells, resulting in NK activation, release of cytolytic granules and consequent cell apoptosis. This is a major killing mechanism of some monoclonal antibodies like rituximab (Rituxan), ofatumumab (Azzera), and others.

Cytokines play a crucial role in NK cell activation. As these are stress molecules released by cells upon viral infection, they serve to signal to the NK cell the presence of viral pathogens in the affected area. Cytokines involved in NK activation include IL-12, IL-15, IL-18, IL-2, and CCL5. NK cells are activated in response to interferons or macrophage-derived cytokines. They serve to contain viral infections while the adaptive immune response generates antigen-specific cytotoxic T cells that can clear the infection. NK cells work to control viral infections by secreting IFNγ and TNFα. IFNγ activates macrophages for phagocytosis and lysis, and TNFα acts to promote direct NK tumor cell killing. Patients deficient in NK cells prove to be highly susceptible to early phases of herpes virus infection.

Tumor-infiltrating NK cells have been reported to play a critical role in promoting drug-induced cell death in human triple-negative breast cancer. Since NK cells recognize target cells when they express non-self HLA antigens (but not self), autologous (patients' own) NK cell infusions have not shown any antitumor effects. Instead, investigators are working on using allogeneic cells from peripheral blood, which requires that all T cells be removed before infusion into the patients to remove the risk of graft versus host disease, which can be fatal. This can be achieved using an immunomagnetic column (CliniMACS). In addition, because of the limited number of NK cells in blood (only 10% of lymphocytes are NK cells), their number needs to be expanded in culture. This can take a few weeks and the yield is donor dependent.

In one embodiment, the invention provides a method of inhibiting tumor growth in a subject including administering to the subject a fusion protein including the sequence set forth in SEQ ID NO:1 and sequences having 90% or greater identity to SEQ ID NO:1, thereby inhibiting tumor growth in the subject.

In one aspect, inhibiting tumor growth includes decreasing tumor cell survival.

In another embodiment, the invention provides a method of increasing survival of a subject having cancer including administering to the subject a fusion protein including the sequence set forth in SEQ ID NO:1 and sequences having 90% or greater identity to SEQ ID NO:1, thereby increasing survival of the subject.

By increasing survival, it is meant that the survival of the subject is increased when the subject is administered the fusion protein of the invention, as compared to the survival in the absence of the administration, or upon administration of another treatment regimen that does not include the fusion protein of the invention.

In an additional embodiment, the invention provides a method of inducing natural killer (NK) mediated antibody-dependent cellular cytotoxicity against a cancer cell in a subject including administering to the subject a fusion protein including the sequence set forth in SEQ ID NO:1 and sequences having 90% or greater identity to SEQ ID NO:1, thereby increasing survival of the subject.

In one aspect, administering to a subject a fusion protein including the sequence set forth in SEQ ID NO:1 and sequences having 90% or greater identity to SEQ ID NO:1 further includes administering to the subject an anti-cancer treatment.

In another aspect, the subject has cancer. In some aspects, the cancer is selected from the group consisting of lung cancer, prostate cancer, multiple myeloma, ovarian cancer and head and neck cancer. In other aspects, cancer cells are B7-H3 expressing cancer cells. In some aspects, the cancer is a treatment resistant cancer.

Presented below are examples discussing the development, characterization and assessment of the efficacy of B7-H3 TriKE molecules, contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Trispecific Development and Characterization

Antigen-specific immunotherapies require overexpression of target antigens on tumor cells with minimal off-tumor expression on normal tissues. Ideally, the antigen displays high expression in a broad spectrum of cancers, making the immunotherapy applicable in a number of settings and basket clinical trials are becoming more popular if broad targets can be identified. B7-H3, a transmembrane costimulatory protein that is a member of the B7 family of checkpoint ligands, has gained interest as a target for immunotherapy. While it is involved both in the context of co-stimulation and inhibition by engaging receptors on T-cells, it has also been shown to contribute to immune evasion through expression on antigen presenting cells, such as macrophages, and tumor cells within the tumor microenvironment. B7-H3 expression is high in many types of cancer but very low in normal tissues. A mouse model, utilizing a B7-H3-targeting CAR T construct that is reactive to mouse cells, has demonstrated anti-tumor responses in the absence of toxicity, further highlighting the safety profile of B7-H3 as a target. Ninety-three percent of ovarian tumors express B7-H3, and expression is associated with advanced stage, high recurrence, and poor survival. Similar findings exist for other types of carcinomas including cancer of the colon, prostate, pancreas, non-small-cell lung cancer, and gastric cancer, indicating that B7-H3 may be a useful marker in cancer biology, progression, and therapy across a range of different cancers. Due to these characteristics, there are currently a number of ongoing clinical trials targeting this antigen in modalities ranging from Fc optimized antibodies (NCT02982941) to CAR T cells (NCT04077866).

Bispecific immune engagers such as blinatumomab have shown impressive clinical success. As a single engineered molecule, one of its single chain variable fragments (scFv) targets cancer cells and the other one targets CD3 on T cells. This creates an immune synapse between T cells and cancer cells, resulting in tumor killing. However, activation and proliferation of T cells can result in cytokine release syndrome, disseminated intravascular coagulation, and nervous system events including encephalopathy and seizures. Thus, the present study aims at selectively engaging natural killer (NK) cells instead of T cells. NK cells are part of the innate immune system, play a major role in tumor surveillance, and have shown potential in a number of studies involving solid tumors and hematologic cancers. Due to these characteristics, a tri-specific killer engager (TriKE) platform consisting of a single chain variable fragment (scFv) targeting CD16, the most potent activating receptor on NK cells, a scFv targeting a tumor associated antigen, and an IL-15 moiety has been designed and described. The inventors have improved on this platform by adding a single domain antibody against CD16, the result of which is better IL-15 activity and overall function. IL-15 is the most critical homeostatic cytokine for NK cell function. It is necessary for NK cell expansion and survival, it can amplify antibody-dependent cellular cytotoxicity (ADCC), it can induce lymphokine-activated killer activity, and it can enhance production of other co-stimulatory mediators like interferon gamma (IFNγ) and tumor-necrosis factor alpha (TNFα).

Described herein is a second-generation TriKE bioengineered with human IL-15 as a modified crosslinker between a humanized camelid anti-CD16 VHH single domain antibody (sdAb) and an anti-B7-H3 scFv, termed cam1615B7-H3. Thus, in a single molecule, two important therapeutic properties were merged: the ability to specifically enhance NK cell expansion with the ability to enhance ADCC. cam1615B7-H3 demonstrated potent and specific induction of NK cell activity against a variety of solid tumors in vitro while also showing potent activity against in a xenogeneic ovarian cancer model. Thus, targeting B7-H3 with a TriKE may have high therapeutic value in the NK-cell-based immunotherapy of a number of solid cancers.

Construction of cam1615B7-H3 TriKEs

Single-domain VHH antibodies derived from camelids are known to offer advantages over conventional VL-VH scFv fragments. The complementary determining regions (CDRs) from a camelid (llama) anti-CD16 were split into a universal, humanized, heavy chain scaffold. This humanized camelid sequence was used to manufacture cam1615B7-H3. A hybrid gene encoding cam1615B7-H3 was synthesized using DNA shuffling and DNA ligation techniques. The fully assembled gene (from 5′ end to 3′ end) encoded a NcoI restriction site; an ATG start codon; anti-human CD16 VHH; a 20 amino acid (aa) segment, PSGQAGAAASESLFVSNHAY (SEQ ID NO:36); human wild-type IL-15; the seven amino acid linker, EASGGPE (SEQ ID NO:37); anti-B7-H3 mAb 376.96 scFv; and a XhoI restriction site. The resulting hybrid gene was spliced into the pET28c expression vector under the control of an isopropyl-D-thiogalactopyranoside (IPTG) inducible T7 promoter. The DNA target gene encoding cam1615B7-H3 was 1527 base pairs. The Biomedical Genomics Center, University of Minnesota (St. Paul, MN, USA) verified the gene sequence and the in-frame accuracy of the construct.

Purification of Protein from Inclusion Bodies

Escherichia coli strain BL21 (DE3) (Novagen, Madison, WI, USA) was used for the expression of proteins after plasmid transfection. Bacterial expression resulted in the sequestering of target protein into inclusion bodies (IBs). Bacteria were cultured overnight in 800 mL Luria broth containing kanamycin (30 mg/mL). When absorbance reached 0.65 at 600 nm, gene expression was induced with Isopropyl β-D-1-thiogalactopyranoside/IPTG (FischerBiotech, Fair Lawn, NJ, USA). Bacteria were harvested after 2 h. After a homogenization step in a buffer solution (50 mM Tris, 50 mM NaCl, and 5 mM EDTA pH 8.0), the pellet was sonicated and centrifuged. Proteins were extracted from the pellet using a solution of 0.3% sodium deoxycholate, 5% Triton X-100, 10% glycerin, 50 mmol/L Tris, 50 mmol/L NaCl, and 5 mmol/L EDTA (pH 8.0). The extract was washed 3 times.

Bacterial expression in inclusion bodies requires refolding. Thus, proteins were refolded using a sodium N-lauroyl-sarcosine (SLS) air oxidation method (20). IBs were dissolved in 100 mM Tris, 2.5% SLS (Sigma, St. Louis, MO USA) and clarified by centrifugation. Then, 50 μM of CuSO4 was added to the solution and then incubated at room temperature with rapid stirring for 20 h for air-oxidization of —SH groups. Removal of SLS was performed by adding 6 M urea and 10% AG 1-X8 resin (200-400 mesh, chloride form) (Bio-Rad Laboratories, Hercules, CA, USA) to the detergent-solubilized protein solution. Guanidine HCl (13.3 M) was added to the solution which was incubated at 37° C. for 2 to 3 h. The solution was diluted 20-fold with refolding buffer, 50 mM Tris, 0.5 M l-arginine, 1 M Urea, 20% glycerol, 5 mM EDTA, pH 8.0. The mixture was refolded at 4° C. for two days and then dialyzed against five volumes of 20 mM Tris-HCl at pH 8.0 for 48 h at 4° C., then eight volumes for 18 additional hours. The product was then purified over a fast flow Q ion exchange column and further purified by passage over a size exclusion column (Superdex 200, GE, Marlborough, MA, USA). Protein purity was determined with sodium dodecyl sulfate polyacrylamide gel electrophoresis (SDS-PAGE) stained with Simply Blue Safe Stain (Invitrogen, Carlsbad, CA, USA).

### Creation and Purification of a B7-H3 Targeting TriKE

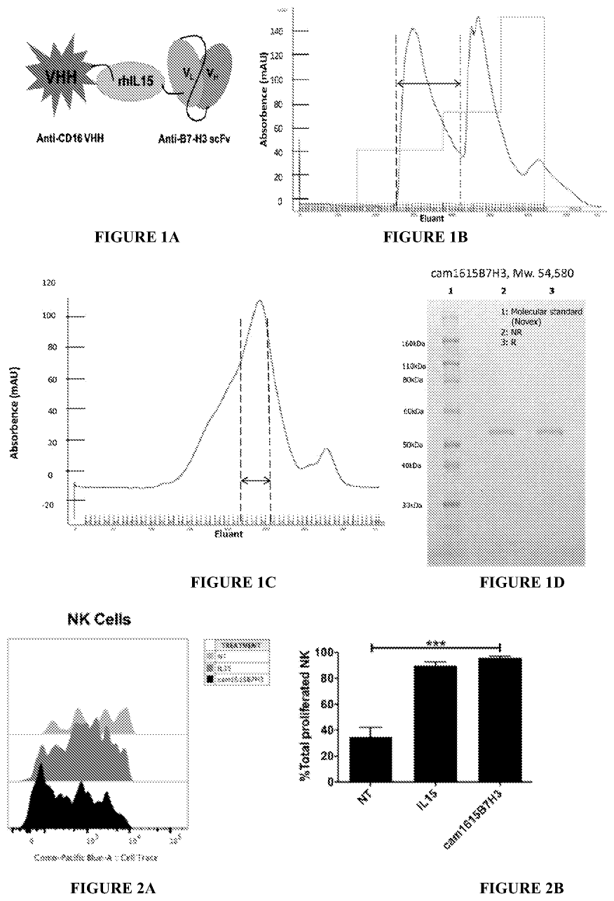

In order to construct a second-generation TriKE capable of both ADCC and NK cell expansion, the existing TriKE platform was modified. A wild-type human IL-15 crosslinker with two modified flanking regions was inserted between two antibody fragments—an N-terminal VHH humanized camelid anti-CD16 fragment and a C-terminal anti-B7-H3 fragment—creating cam1615B7-H3. FIG. 1A shows a schematic of the B7-H3 TriKE construct. B7-H3 TriKE includes single chain variable fragments from camelid nanobodies (cam) targeting CD16 and B7-H3 joined by IL-15 and two flexible linker regions to form a single peptide with molecular weight of −46 kDa. This differs from the BiKE, which consists of camCD16 and camB7-H3 with a single flexible linker region to form a single peptide of approximately 35 kDa. NK cell-mediated target lysis is directed towards B7-H3-expressing tumor cells via formation of a direct physical link by the B7-H3 TriKE or BiKE. IL-15 then stimulates the NK cells. FIG. 1B shows the absorbance tracing from the FFQ ion exchange column as the first phase of the purification with the eluant collected in 8-mL aliquots shown on the abscissa of the graph. The double-sided arrow shows the collection peak as drug exits the column. FIG. 1C shows the absorbance tracing from the second purification phase, size exclusion chromatography (SEC). The first peak off the column was collected and the various drug containing fractions were pooled and analyzed using SDS-PAGE with Comassie Blue staining for the presence of a uniform product (FIG. 1D). The final product was greater than 90% pure with a molecular weight of about 55 kDa; the predicted molecular weight was 54.58 kDA. As with other TriKE molecules, this TriKE is expected to have a rapid clearance profile due to its size, with EC50 ranges in the order of a couple of hours.

cam1615B7-H3 TriKE Induces Potent and Specific NK Cell Proliferation

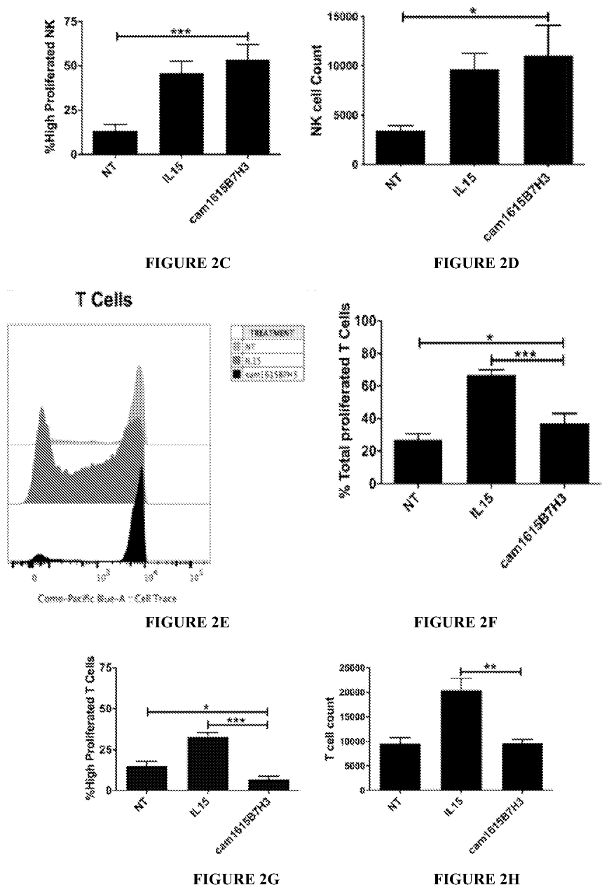

The wild-type IL-15 moiety in the cam1615B7-H3 TriKE was designed to induce targeted delivery of a proliferative signal to NK cells. To test this, proliferation assays evaluating dilution of a CellTrace dye over a 7-day period were carried out on PBMCs treated with no treatment (NT), monomeric rhIL-15 (IL15), or the TriKE (cam1615B7-H3). At the end of the seven days, cells were harvested and proliferation was evaluated by gating on CD56+CD3− cells. While no treatment (NT) resulted in low proliferation with low NK cell numbers, the cam1615B7-H3 induced an overall increase in proliferation that was similar in amplitude to that induced by rhIL-15 (FIGS. 2A-2D), with no significant differences between those two groups. Since IL-15 acts on both NK cells and T cells, specificity was evaluated next by gating on T cells (CD56-CD3+). Minimal T cell proliferation was seen in the TriKE treatment group in contrast to rhIL-15, which induced robust proliferation of T cells (FIGS. 2E AND 2F) clearly showing that the cam1615B7-H3 TriKE IL-15 delivery was more restricted to NK cells. This differential was particularly notable in robustly proliferating populations (past three divisions), where the cam1615B7-H3 TriKE showed significantly less proliferation than the untreated group (FIG. 2G), while T cell numbers did not differ between the untreated and TriKE-treated groups (FIG. 2H). This data indicates that the cam16 engager in the cam1615B7-H3 TriKE is specifically delivering IL-15 to NK cells and not T cells.

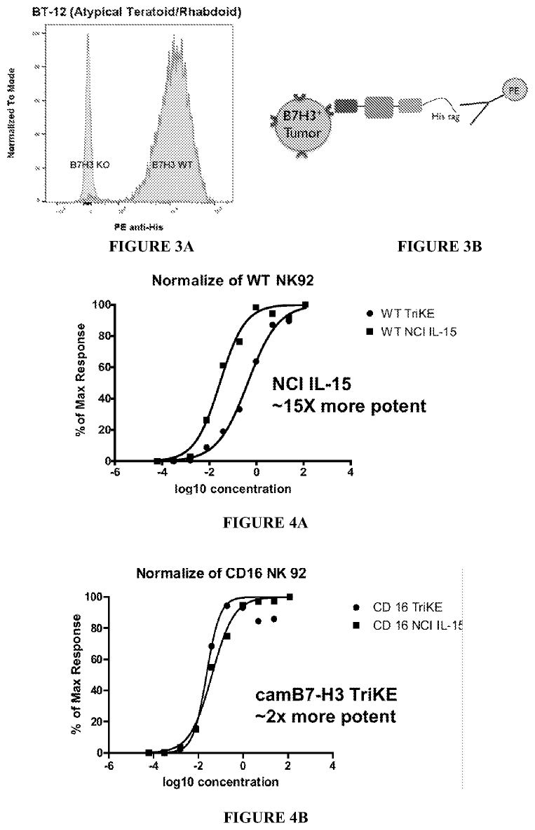

As illustrated in FIGS. 3A and 3B, the camB7-H3 TriKE of the invention has demonstrated a strong binding specificity against WT B7-H3. BT-12 pediatric brain tumor lines highly express B7-H3 (WT). A B7-H3 KO BT-12 cell line was produced using CRISPR (Theruvath et al). Similar specificity was noted using Raji (negative B7-H3) and prostate cancer cell lines C4-2 (positive B7-H3) and multiple other lines. B7-H3 BiKE had similar binding with positive and negative cell lines (Data not shown).

NK-92 cells without or with CD16 were incubated for 48 hours with dilutions of NCI IL-15 and GTB-5550. Metabolic activity was then measured using resazurin (n=4). As Shown in FIGS. 4A-4B the TriKE molecule was found twice as potent as NCI IL-15 in CD16+ NK-92.

### Example 2

### General Material and Methods

### Cancer Cell Lines and Antibody

MA-148 (established locally at the University of Minnesota) is a human epithelial high-grade serous ovarian carcinoma cell line. For in vivo experiments, lines were transfected with a luciferase reporter construct using Invitrogen's Lipofectamine Reagent and selective pressure applied with 10 μg/mL of blasticidin. Ovarian carcinoma cell lines OVCAR5 and OVCAR8 were obtained from the DTP, DCTD Tumor Repository sponsored by the Biological Testing Branch, Developmental Therapeutics Program, National Cancer Institute (NCI), National Institutes of Health (NIH, Frederick, MD, USA). Other cell lines were obtained from the American Type Culture Collection including OVCAR3 (ovarian), C4-2 (prostate), DU145 (prostate), LNCaP (prostate), PC-3 (prostate), A549 (lung), NCI-H322 (lung), NCI-H460 (lung), and Raji cells (Burkitt's lymphoma). With the exception of Raji cells, used as a negative control, all lines express high levels of B7-H3. Lines were maintained in RPMI 1640 medium supplemented with 10-20% fetal bovine serum (FBS) and 2 mmol/L L-glutamine. Lines were incubated at a humidified atmosphere containing 5% C02 at a constant 37° C. When adherent cells were more than 90% confluent, they were passaged using trypsin-EDTA for detachment. For the cell counts a standard hemocytometer was used. Only those cells with a viability >95% were used for the experiments. The sequence for the monoclonal antibody scFv fragment 376.96 was obtained by Dr. Ferrone and used to construct the TriKE.

### Cell Products

Peripheral blood mononuclear cells (PBMCs) were obtained from normal volunteers or patients after consent was received, and institutional review board (IRB) approval was granted (protocols 9709M00134 and 1607M91103), in compliance with guidelines by the Committee on the Use of Human Subjects in Research and in accordance with the Declaration of Helsinki. For in vivo studies, fresh PBMCs were magnetically depleted three times (i.e., three passthroughs across the magnet) of CD3 and CD19-positive cells, according to the manufacturer's recommendations (STEMCELL Technologies, Cambridge, MA, USA), to generate an NK-cell-enriched product. Ovarian cancer specimens (ascites) were collected in women diagnosed with advanced-stage ovarian or primary peritoneal carcinoma at time of primary debulking surgery. For prostate cancer, blood was obtained from two patients with metastatic castration resistant prostate cancer and one patient with metastatic hormone sensitive prostate cancer. For lung cancer, blood was obtained from seven unresectable lung cancer patients at the time of diagnosis, prior treatment. Cells were pelleted, lysed for red blood cells, cryopreserved in 10% DMSO/90% FBS, and stored in liquid nitrogen.

### NK Cell Expansion Via IL-15 Stimulation

To measure the ability of the TriKE to specifically induce NK cell expansion via the IL-15 moiety, PBMCs from healthy donors were labeled with CellTrace Violet Proliferation Dye (Invitrogen, Carlsbad, CA, USA) according to kit specifications. After staining, cells were cultured with TriKEs at noted concentrations, or equimolar concentrations of controls, and incubated in a humidified atmosphere containing 5% CO2 at 37° C. for seven days. Cells were harvested, stained for viability with Live/Dead reagent (Invitrogen, Carlsbad, CA, USA) and surface stained for anti-CD56 PE/Cy7 (Biolegend, San Diego, CA, USA) and anti-CD3 PE-CF594 (BD Biosciences, Franklin Lakes, NJ, USA) to gate on the viable CD56+ CD3− NK cell population or the CD56-CD3+ T cell population. Data analysis was performed using FlowJo software (FlowJo LCC, version 7.6.5, Ashland, OR, USA).

### Evaluation of Cytotoxicity and NK Cell Activation

ADCC was measured in a flow cytometry assay by evaluating degranulation via CD107a (lysosomal-associated membrane protein LAMP-1) and intracellular IFNγ production. Upon thawing, normal donor and patient-derived PBMCs or ascites cells were rested overnight (37° C., 5% CO2) in RPMI 1640 media supplemented with 10% fetal calf serum (RPMI-10). The next morning, they were suspended with tumor-target cells or media after washing twice with RPMI-10. Cells were then incubated with TriKEs or controls for 10 min at 37° C. Fluorescein isothiocyate (FITC)-conjugated anti-human CD107a monoclonal antibody (BD Biosciences, San Jose, CA, USA) was then added. Following an hour 37° C. incubation, GolgiStop (1:1500, BD Biosciences) and GolgiPlug (1:1000, BD Biosciences) were added for 3 h. After washing with phosphate buffered saline, the cells were stained with PE/Cy 7-conjugated anti-CD56 mAb, APC/Cy 7-conjugated anti-CD16 mAb, and PE-CF594-conjugated anti-CD3 mAb (BioLegend, San Diego, CA, USA). Cells were incubated for 15 min at 4° C., washed, and fixed with 2% paraformaldehyde. Cells were then permeabilized using an intracellular perm buffer (BioLegend) to evaluate production of IFNγ through detection via aBV650 conjugated anti-human IFNγ antibody (BioLegend). Samples were washed and evaluated in an LSRII flow cytometer (BD Biosciences, San Jose, CA, USA).

### Real-Time Tumor-Killing Assay

Tumor killing was evaluated in real-time using the IncuCyte platform. Magnetic-bead-enriched CD3−CD56+ NK effector cells were plated into 96-well flat clear-bottom polystyrene tissue-culture-treated microplates (Corning, Flintshire, UK) along with NuclightRed stably expressing OVCAR8 cells at a 2:1 effector:target ratio. Caspase-3/7 green dye (Sartorious, Ann Arbor, MI, USA) was added to pick up dying cells that have not yet lost NuclightRed fluorescence. Noted treatments were then added at a 30 nM concentration, and the plate was placed in an IncuCyte ZOOM® platform housed inside a cell incubator at 37° C./5% C02. Images from three technical replicates were taken every 15 min for 48 h using a 4× objective lens and then analyzed using IncuCyte™ Basic Software v2018A (Sartorious). Graphed readouts represent percentage live OVCAR8 targets (NuclightRed+Caspase-3/7−), normalized to live targets alone at the starting (0 h) time point.

### Mass Cytometry (CyTOF)

For mass cytometry (CyTOF) studies, PBMCs were incubated alone or with OVCAR8s at a 2:1 ratio+/−cam1615B7-H3 (30 nM) for 24 h. After harvesting samples, cells were counted, and viability was measured using trypan blue exclusion. Two hundred thousand cells from each donor were aliquoted into 5-mL polystyrene U-bottom tubes for barcoding and CyTOF staining. Cells were stained with Cisplatin (Fluidigm Product #201064, San Francisco, CA, USA), followed by barcoding using the Cell-ID 20-Plex Pd Barcoding Kit (Fluidigm Product #201060). After barcoding, all cells were combined into a single 5-mL polystyrene U-bottom tube and incubated in the surface marker antibody cocktail for 30 min at 4° C.

Following surface staining, cells were then fixed using 2% PFA. For intracellular staining, cells were permeabilized by incubation with Triton X 0.1% for 5 min at room temperature, followed by incubation with intracellular antibody cocktail for 30 min at 4° C. Stained cells were then incubated overnight with Cell-ID Intercalator (Fluidigm Product #201192A). The following morning cells were washed and run on the CyTOF 2 instrument. Wash steps were completed using either Maxpar PBS (Fluidigm Product #201058), Maxpar Cell Staining Buffer (Fluidigm Product #201068), or Millipure Water at 1600 RPM for 4 min. For custom tagged antibodies: Conjugation of heavy metals to a specific ScFv is conducted using the Maxpar antibody labeling kit (Fluidigm). The protocol involves partial antibody reduction using 0.5 M TCEP: Pierce Bond-Breaker TCEP Solution (Thermo Scientific Product #77720, Waltham, MA, USA), as well as comprehensive buffer exchange using centrifugal filter units of both 3 kDa and 50 kDa size (Millipore Product #UFC500396, UFC505096, Burlington, MA, USA). After conjugation of the antibody, yield is measured, and the final reagent is stored in antibody stabilizer (Boca Scientific Product #131 000, Westwood, MA, USA). The reagent is then titrated and verified against known flow cytometry antibodies. Data from the three donors was concatenated. FCS file concatenation was completed with a combination of Cytobank and Flowjo. All Visne analyses were carried out in Cytobank.

### In Vivo Mouse Study and Imaging

MA-148-Luc ovarian cancer cells were incorporated into a previously described NK cell xenogeneic mouse model system. NSG mice (NOD.Cg-Prkdcscid Il2rgtm1Wjl/SzJ, n=5/group) were injected IP with 2.0×105 MA-148-luc cells and then three days later conditioned with low-dose total body irradiation (225 cGy). The following day, all groups received highly enriched NK cells (PBMC magnetically CD3 and CD19 depleted), equivalent to 1 million NK cells/mouse, and were started on the drug regimen. A single course of treatment consisted of an IP injection of 30 g of TriKE or 5 μg rhIL-15 given every day of the week (Monday-Friday) for three weeks. MA-148-luc cells are a subline of MA-148 that have been transfected with a luciferase reporter gene, allowing for imaging of the mice each week to determine their bioluminescent activity and to monitor tumor progression. Briefly, mice were injected with 100 μL of 30 mg/mL luciferin substrate 10 min prior to imaging and then anesthetized via inhalation of isoflurane gas (25). Mice were then imaged using the Xenogen Ivis 100 imaging system and analyzed with Living Image 2.5 software (Xenogen Corporation, Alameda, CA, USA). At the end of the experiment (day 21), all the animals were sacrificed, and postmortem peritoneal lavages were performed to analyze human NK cell content by flow cytometry. Animal imaging and analysis was performed at the University of Minnesota Imaging Center. Mouse studies were carried after approval (protocol 1908-37330A) from the Institutional Animal Care and Use Committee (IACUC) at the University of Minnesota and in compliance with their guidelines.

### Statistical Analysis

GraphPad PRISM 8 (GraphPad Prism Software, Inc., San Diego, CA, USA) was used to create all statistical tests. For all in vitro studies, one-way ANOVA with repeated measures was used to calculate significance in comparisons to the cam1615B7-H3 group. For mouse studies, two-way ANOVA was used to calculate significance in the longitudinal study, while one-way ANOVA was used to calculate the significance in differences in radiance at the day-21 timepoint. An unpaired t test was used to evaluate differences in cell counts and MFI. Bars represent mean±SEM. Statistical significance is displayed as * p<0.05, ** p<0.01, ***p<0.001, and **** p<0.0001.

### Example 3

### Hematological Cancer Cells

The efficacy of the H7-B3 TriKE molecules of the invention was assessed in several hematological cancer cell lines. Unless described otherwise, the methods used are as described in Example 2.

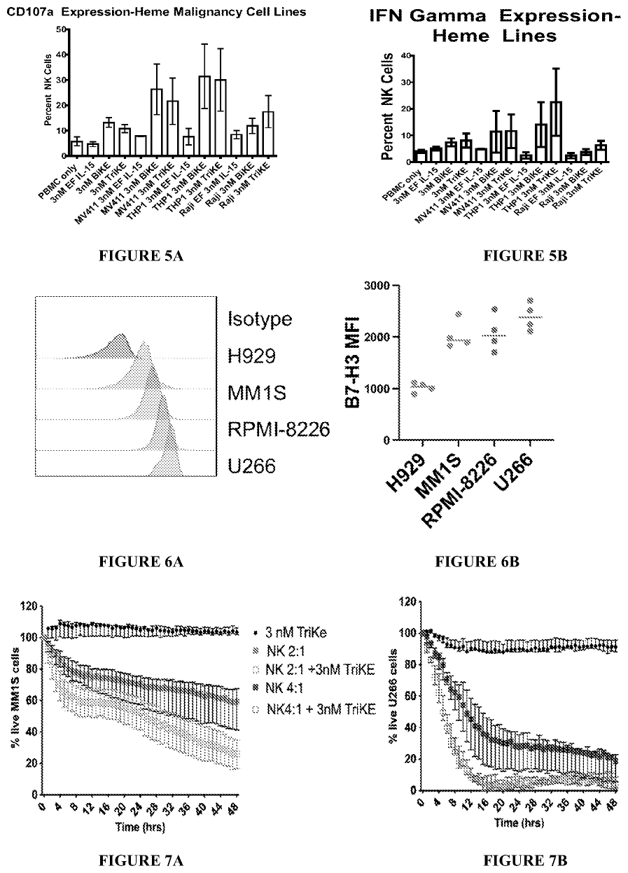

As illustrated in FIGS. 5A-5B, functional assays were conducted with hematologic malignancy cell lines with varying levels of B7-H3 expression from none to very high levels. NK cell activation was measured using CD107a and IFN gamma as measured by flow cytometry (n=3), IncuCyte, or xCelligence assay.

The analysis was then focused on multiple myeloma. B7-H3 (CD276) expression on myeloma is associated with decreased progression free survival, it exhibits low expression on healthy tissue, and it is expressed on myeloid derived suppressor cells (MDSC), which promote myeloma growth.

As shown in FIGS. 6A-6B, high expression of B7-H3 was found on the myeloma lines RPMI-8226, U266, and MM1S and relatively low expression on H929 by flow cytometry.

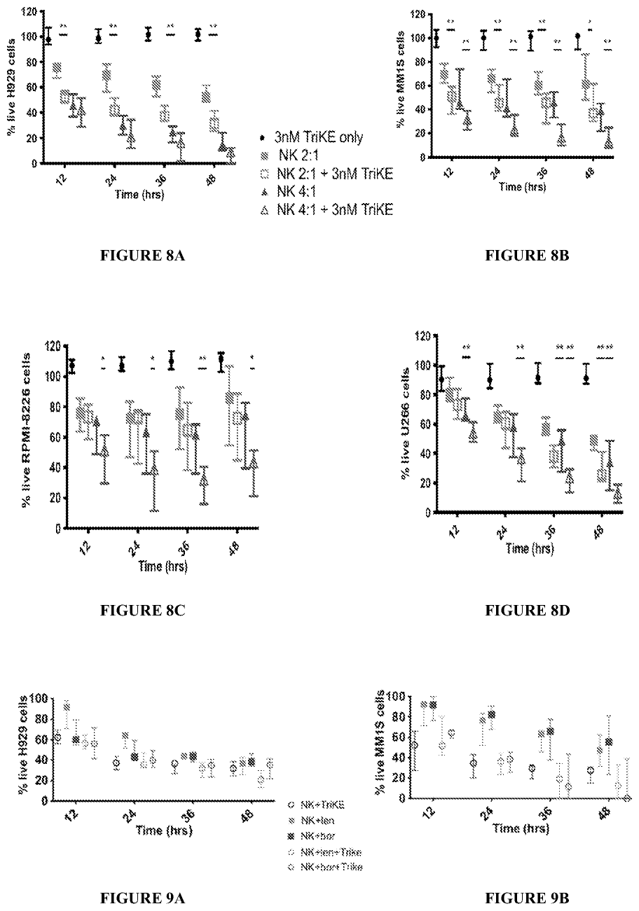

The ability of peripheral blood NK cells with or without B7-H3-TriKE to kill myeloma cells was compared in live imaging IncuCyte Zoom assays with escalating doses of TriKE. Maximal killing occurred with 3 nM concentration. A statistically significant increase in NK cell mediated killing of all four myeloma lines when 3 nM B7-H3-TriKE was added was found. Against U266 and MM1S, B7-H3-TriKE significantly enhanced killing at effector:target (E:T) ratios of 2:1 and 4:1. RPMI-8226 showed relatively high resistance to NK Cell cytotoxicity but B7-H3-TriKE enhanced killing at E:T of 4:1. H929 cells were more potently killed in the presence of B7-H3-TriKE at E:T of 2:1 but there was no difference in killing at E:T 4:1 likely due to high natural cytotoxicity in both groups (see FIGS. 7A-7B and 8A-8D).

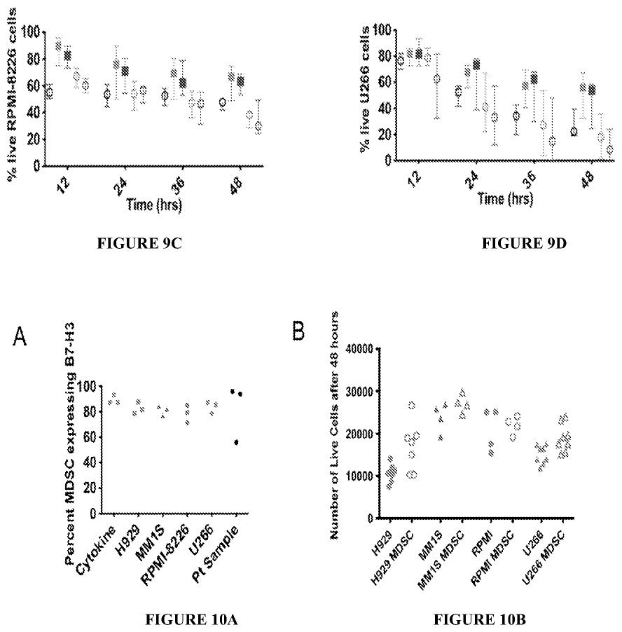

The efficacy of B7-H3-TriKE with the proteasome inhibitor bortezomib (10 nM) and the immunomodulatory drug lenalidomide (5 μM) was also tested. Cytotoxicity curves were compared by repeated measures ANOVA and performed in triplicate. Combination therapy with B7-H3-TriKE, NK cells, and lenalidomide showed synergistic killing of H929 cells after 48 hours of live cell imaging (p=0.047) but combination with bortezomib did not further enhance killing compared to NK cells and TriKE alone (FIG. 9A). Both lenalidomide and bortezomib showed a trend toward improved killing against MM1S when given with NK cells and B7-H3 TriKE but it did not reach statistical significance (FIG. 9B). Combination therapy with B7-H3-TriKE, NK cells, and lenalidomide or bortezomib showed synergistic killing of RPMI-8226 cells after 48 hours of live cell imaging (p<0.001 and 0.015 respectively) (FIG. 9C). Bortezomib combined with B7-H3-TriKE and NK cells enhanced killing in U266 cells (p=0.037) (FIG. 9D).

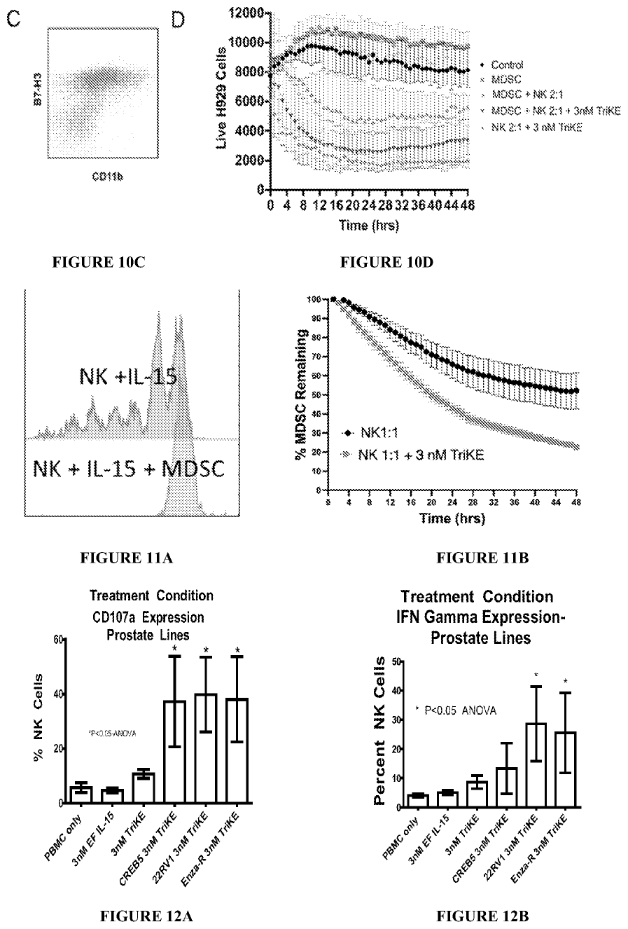

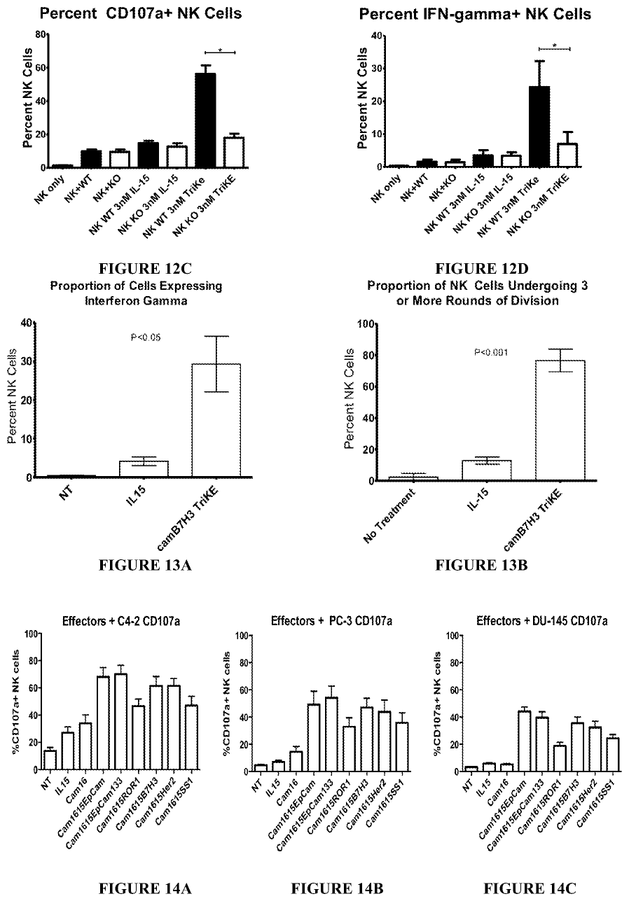

MDSC were developed from CD33+ myeloid cells from healthy donors using IL-6 and GM-CSF or by incubating them with myeloma cells at 1:100 ratio for seven days. MDSC (CD14+CD11b+) exhibited high expression of B7-H3 (FIG. 10A). MDSC were also isolated from the bone marrow aspirates of three newly diagnosed myeloma patients and exhibited 56-95 survival (aspirates were processed with lysis buffer and stained for CD14, CD11b, and B7-H3. Shown is a flow cytometry plot of live, CD14+ cells (FIG. 10C). MDSC were incubated with myeloma cells and growth was measured over 48 hours by live cell imaging (FIG. 10B). Addition of MDSC to cytotoxicity assays enhanced myeloma cell growth but was overcome by B7-H3 TriKE and NK cells (FIG. 10D). B7-H3-TriKE significantly enhanced NK cell mediated killing of myeloma cells, even in the relatively low B7-H3-expressing H929 line. This also shows it can reverse MDSC-induced myeloma growth.

Since MDSC expressed B7-H3 they were co-cultured MDSC with NK at E:T of 1:1 and killing with and without B7-H3 TriKE was compared (see FIGS. 11A and 11B).

### Example 4

### Efficacy of H7-B3 TriKE in Prostate Cancer

The efficacy of the H7-B3 TriKE molecules of the invention was assessed in several prostate cancer cell lines. Unless described otherwise, the methods used are as described in Example 2.

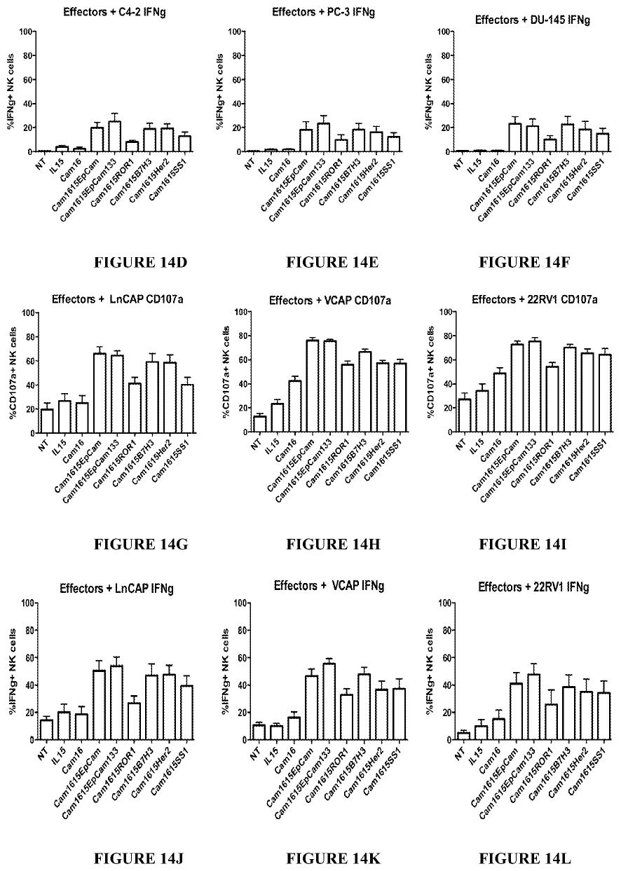

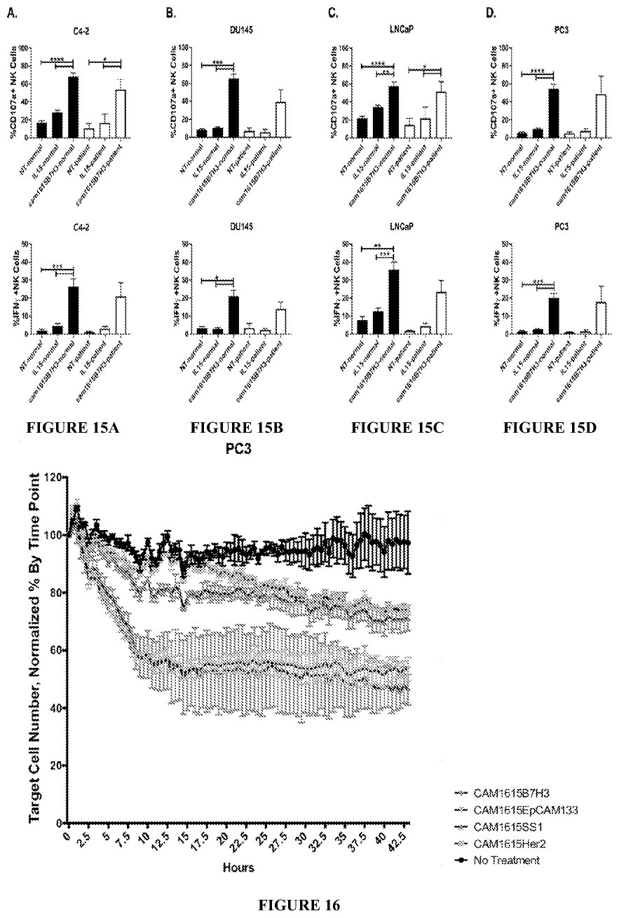

As illustrated in FIGS. 12A-12D, 14A-14L and 15A-15D cam1615B7-H3 TriKE targets prostate cancer. The ability of the cam1615B7-H3 TriKE to improve NK cell activity against prostate cancer was tested. All of the prostate cancer cell lines tested expressed B7-H3. For these tests, normal donor PBMCs and PBMCs obtained from metastatic prostate cancer patients were used. While metastatic prostate cancer patients displayed a slight decrease in NK cell activity, when compared to normal donors, the cam1615B7-H3 TriKE enhanced degranulation and IFNγ production, in both normal donor and patient NK cells, against C4-2, DU145, LNCaP and PC3 prostate cancer adenocarcinoma cell lines when compared to the controls (FIGS. 14A-14G, 14J and 15A-15D). The individual cam16 VHH or anti-B7-H3 scFv components did not induce increased NK cell activation against C4-2s. Thus, the data indicates that the cam1615B7-H3 TriKE has promise in NK cell immunotherapy within the prostate cancer setting and shows that the NK cell function can be rescued on patients who require novel interventions due to poor outcomes with current therapeutic approaches. The signal induced by the TriKE with prostate cancer cells was stronger than that induced by a strong natural cytotoxicity signal and was specific to B7-H3.

As shown in FIG. 13A-13B cam1615B7-H3 TriKE were more potent at inducing NK function than IL-15 alone and were also more potent at inducing NK cell proliferation, as compared to IL-15 alone.

As illustrated in FIG. 16, tumor killing of PC-3 cells was evaluated in real-time using the IncuCyte platform, which emphasized the enhanced efficacy of the B7-H3 TriKE molecules to induce prostate cancer cell death.

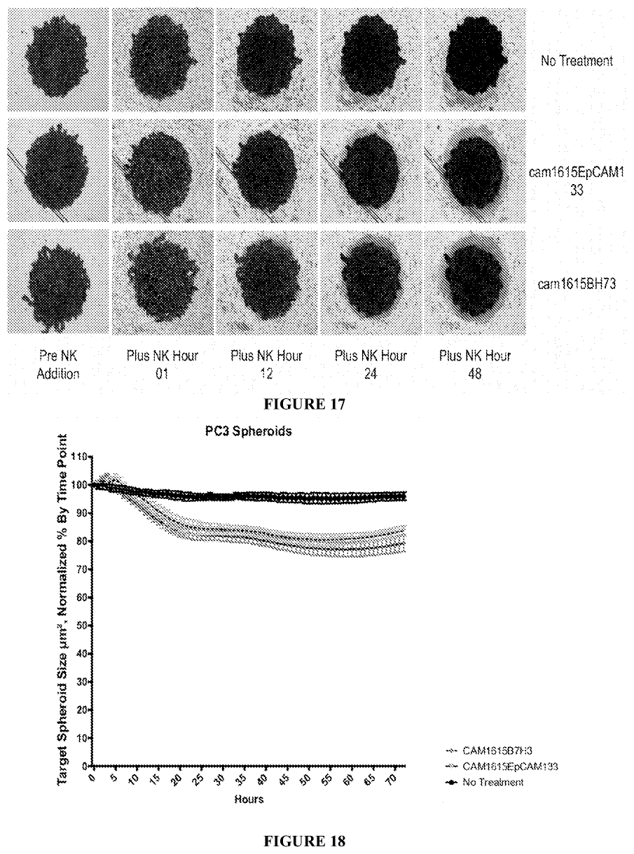

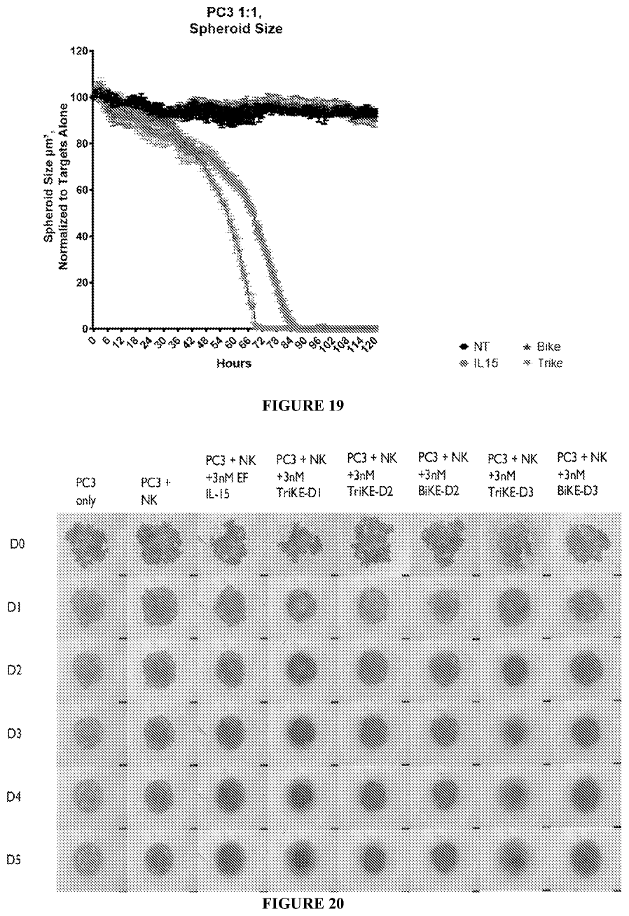

As shown in FIGS. 17-20, the B7-H3 TriKE molecules were also able to reduce PC-3 spheroid size over time.

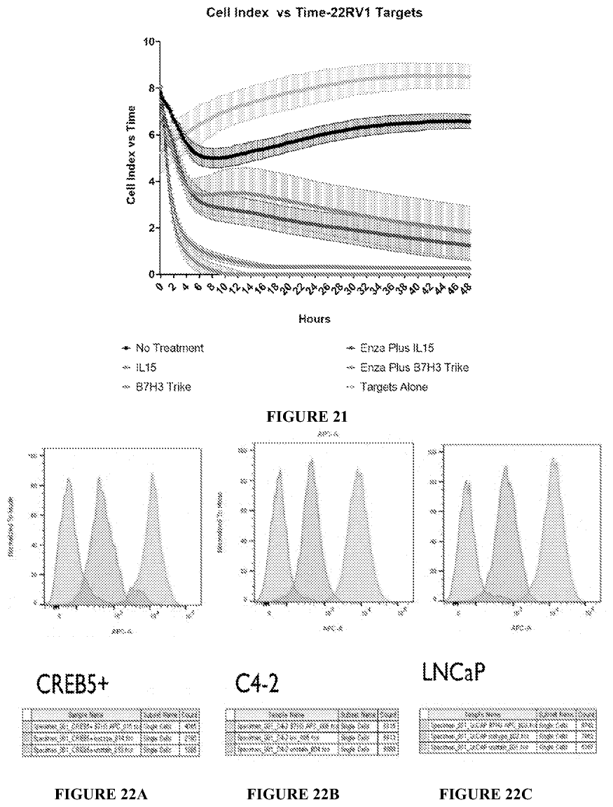

As illustrated in FIG. 21, the killing of prostate cancer cells by the B7-H3 TriKE molecules was demonstrated to happen rapidly (within a couple of hours) after initiation of the treatment.

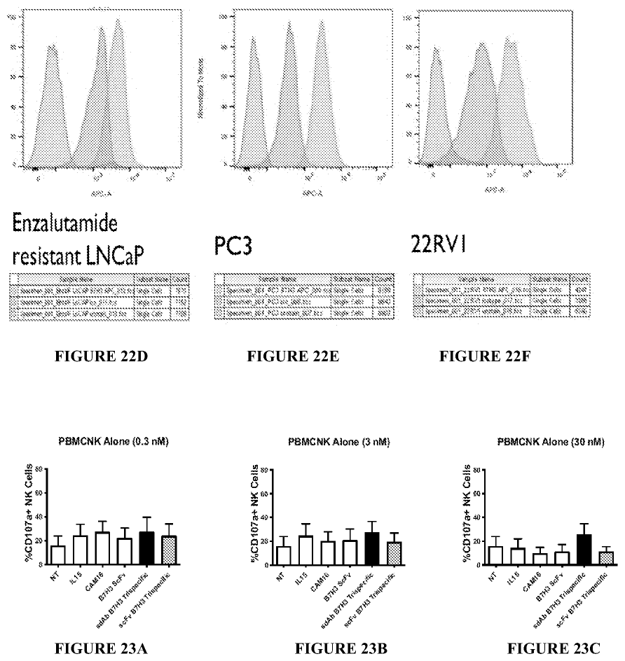

Various enzalutamide resistant prostate cancer cells were phenotyped for their expression of B7-H3. As illustrated in FIGS. 22A-22F all the cell lines tested expressed B7-H3.

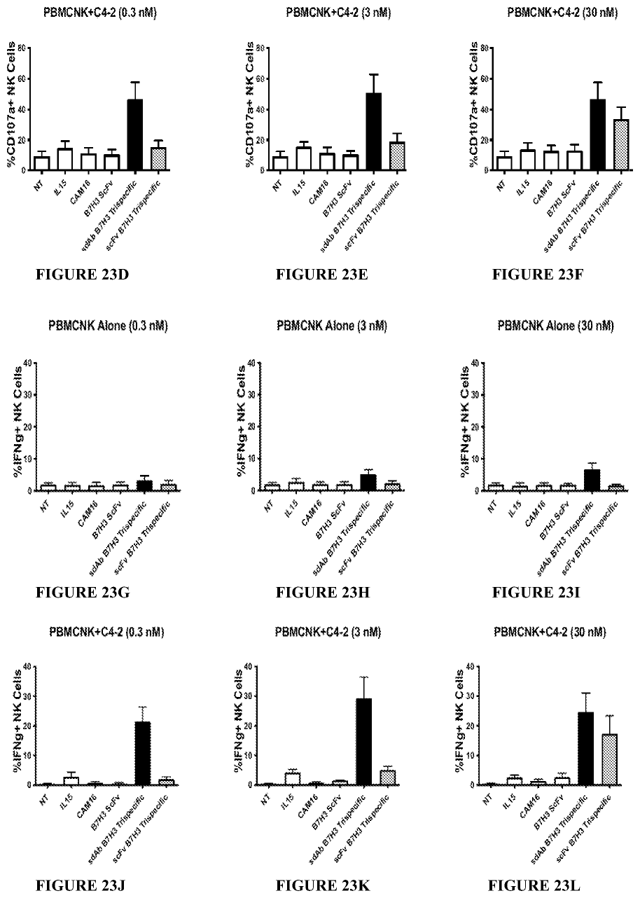

As shown in FIG. 23A-23L, camB7-H3 TriKE were found to induce activity against prostate cancer cells over a broader dynamic range than previous scFv version, as evaluated by measuring the percent of CD107a+ and IFNγ+ NK cells.

### Example 5

### Efficacy of H7-B3 TriKE in Lung Cancer

The efficacy of the H7-B3 TriKE molecules of the invention was assessed in several lung cancer cell lines. Unless described otherwise, the methods used are as described in Example 2.

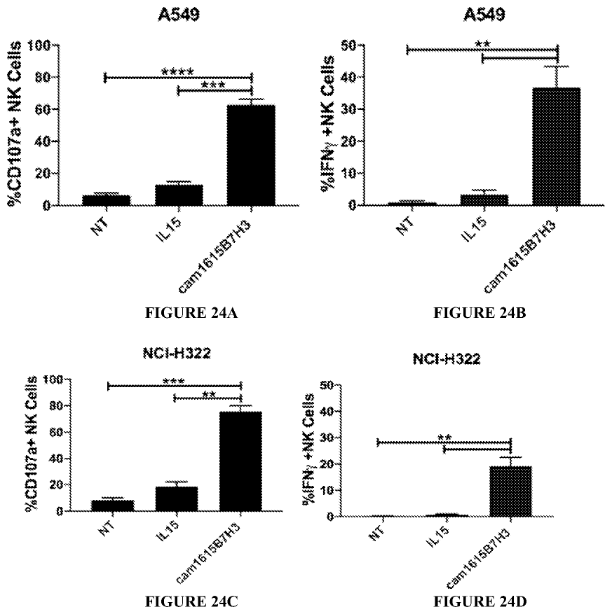

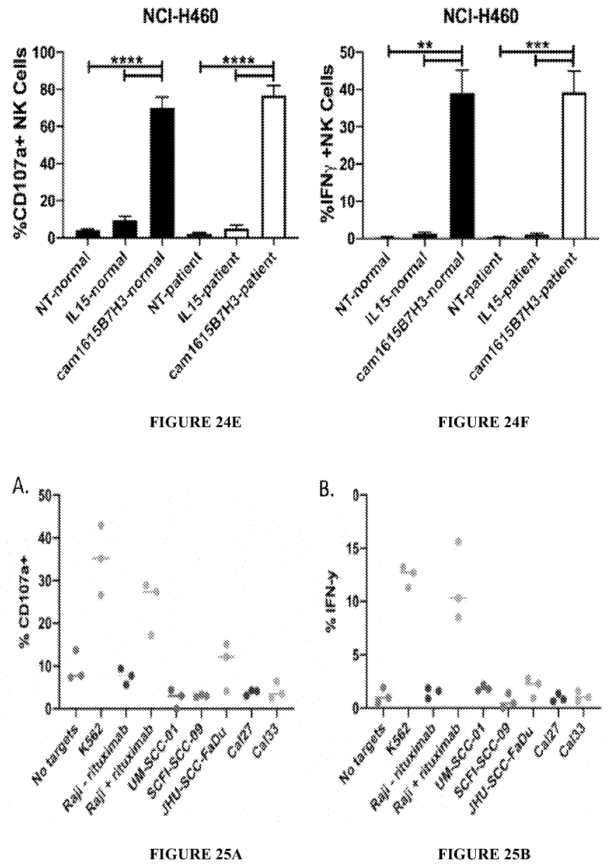

As illustrated in FIGS. 24A-24F, cam1615B7-H3 TriKE targets lung cancer. The ability of cam1615B7-H3 TriKE to improve NK cell activity against B7-H3-expressing lung cancer was tested on normal donor PBMCs incubated with A549 and NCI-H322, two non-small cell lung cancer adenocarcinoma lines (FIGS. 24A-24D). In both instances the cam1615B7-H3 TriKE significantly and robustly improved NK cell activation when compared to controls. Individual cam16 VHH or anti-B7-H3 scFv components were tested and showed no background NK cell activity against A549s. Normal donor PBMCs as well as PBMCs from patients with newly diagnosed unresectable lung cancer, prior to any therapy, were incubated with NCI-H460 cells, a large cell lung cancer cell line. As the data clearly shows, cam1615B7-H3 treatment strongly increased NK cell function, on both normal donor and patient samples, against large cell lung cancer when compared to controls (FIGS. 24E-24F). The TriKE-mediated induction of NK cell degranulation and IFNγ production against lung cancer cells was higher than that seen when NK cells are incubated with K562 targets alone. Activation against lung cancer cell lines was specific to B7-H3 expression as it was higher than activation by B7-H3− Raji cells. Thus, the data indicates that the cam1615B7-H3 TriKE has broad B7-H3-specific activity against a number of solid tumor targets.

### Example 6

### Efficacy of H7-B3 TriKE in Head and Neck Cancer

The efficacy of the H7-B3 TriKE molecules of the invention was assessed in several head and neck cancer cell lines. Unless described otherwise, the methods used are as described in Example 2.

Worldwide, Head and Neck Squamous Cell Carcinomas (HNSCC) account for about 900,000 cases and 400,000 deaths. In some settings, like Fanconi anemia (FA), patients receive curative treatments (allogeneic stem cell transplantation), only to develop HNSCC in early adulthood at a high rate of incidence. Current treatment strategies for non-FA HNSCC patients include surgery, chemotherapy and radiotherapy. However, these are not viable treatment options for FA HNSCC patients due to their low tolerance for the high toxicity levels of chemotherapy and radiation. Therefore, there is a critical need for novel and targeted therapeutic interventions for the treatment of FA HNSCC patients.

B7-H3, a checkpoint member of the B7 and CD28 families, is overexpressed on several solid tumors but is absent or not expressed on healthy tissues. It is a promising target for immunotherapy, and recent basket trials, particularly in prostate cancer, have demonstrated strong clinical signals. Here the ability of a tri-specific killer engager (TriKE) that includes a B7-H3 targeting component, was developed and tested to direct NK cell killing to B7-H3-expressing Head and Neck cancer targets. This TriKE molecule includes an NK cell engaging domain containing a humanized camelid nanobody against CD16, a camelid nanobody against B7-H3 and a wild type IL-15 sequence between the two engagers. B7-H3 expression was assessed by flow cytometry on wild-type HNSCC cells and a paired version with a CRISPER KO of the FANCA gene and it was determined that the KO had no effect on B7-H3 expression. Thus, the TriKE activity against HNSCC should be present on both normal HNSCC and FA-HNSCC settings.

NK cell responses against HNSCC lines in the presence of the B7-H3 TriKE were assessed through either flow cytometry based functional assays, to evaluate NK cell degranulation and cytokine secretion, or IncuCyte imaging assays, to directly assess target killing. NK cell degranulation and IFN-gamma production of B7-H3 TriKE-treated samples were higher compared to that of control samples treated with B7-H3 single domain or IL-15 alone. B7-H3 TriKE also induced more HNSCC target cell killing by NK cells compared to treatment with the B7-H3 single domain or IL-15 alone irrespective of the FANCA gene, both in 2D and 3D IncuCyte imaging assays. Ongoing experiments will evaluate the functionality and efficacy of the B7-H3 TriKE in vivo. Taken together, this data shows that B7-H3 TriKE is able to drive NK cell activity against B7-H3− CD16, a camelid nanobody against B7-H3 expressing HNSCC cells, which presents potential for a B7-H3-targeted TriKE to be used to be implemented clinically to treat HNSCC or FA-HNSCC patients.

As shown in FIGS. 25A-25B, frozen PBMCs (N=3) from healthy donors were incubated for 5 hours with 5 HNSCC cell lines: UM-SCC-01, SFCI-SCC-07, JHU-SCC-FaDu, Cal27 and Cal33 to evaluate CD107a expression (as a marker for degranulation) and intracellular IFN-γ production. HNSCC cell lines did not induce NK cell cytolytic function without treatment.

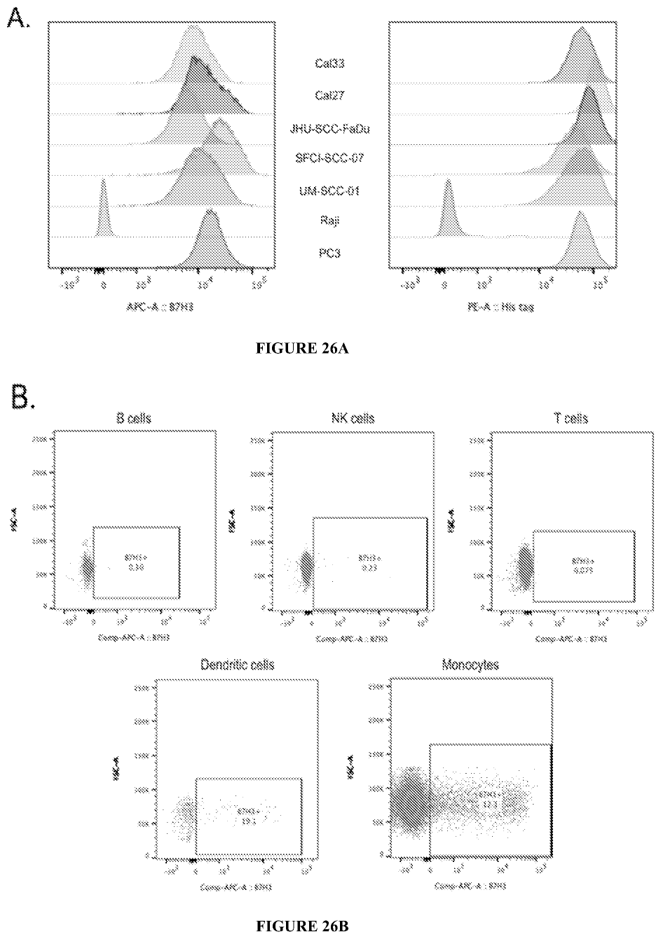

As shown in FIGS. 26A-26B, 5 HNSCC cell lines were assessed for B7-H3 expression and binding affinity with B7-H3 single domain via flow cytometry and PBMCs from a healthy donor were assessed for B7-H3 expression by flow cytometry. B7-H3 is highly expressed on HNSCC but not on healthy immune cells.

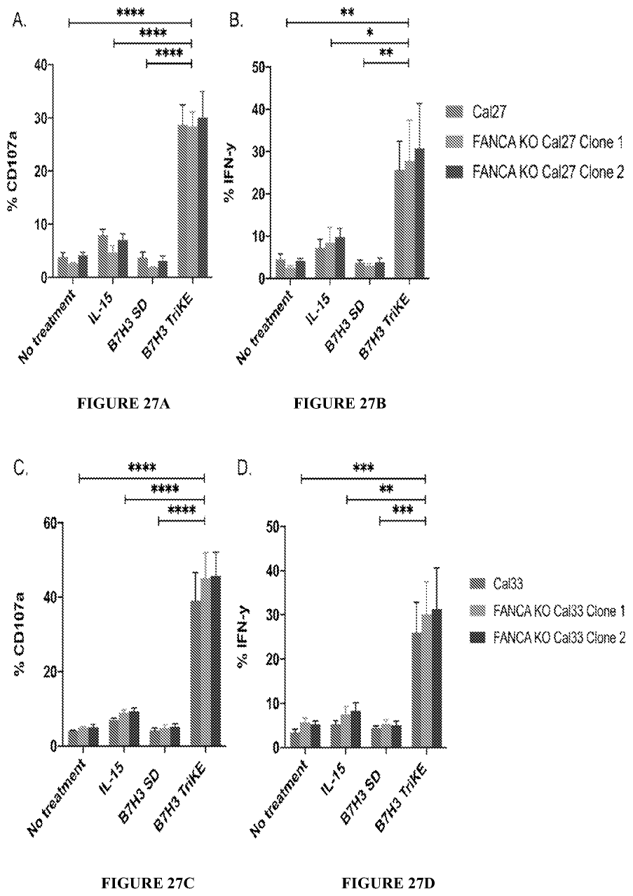

As shown in FIG. 27A-27D, B7-H3 TriKE induced NK cell activity against HNSCC. Frozen PBMCs (N=3) from healthy donors were incubated for 5 hours with (A-B) Cal27 trio and (B-C) Cal33 trio (each trio consisting of a HNSCC WT line and 2 clones of HNSCC FANCA KO lines) in different treatments: no treatment or 3 nM IL-15, MOPC, B7-H3 SD and B7-H3TriKE to evaluate CD107a expression (as a marker for degranulation) and intracellular IFN-γ production. Error bars indicate standard error of mean, and statistical significance was determined as *p<0.05, **p<0.01, ***p<0.001 and ****p<0.0001

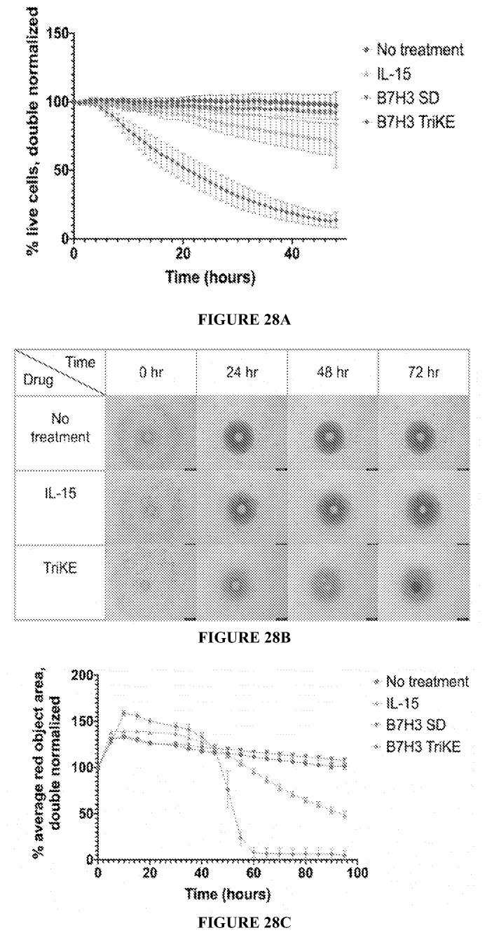

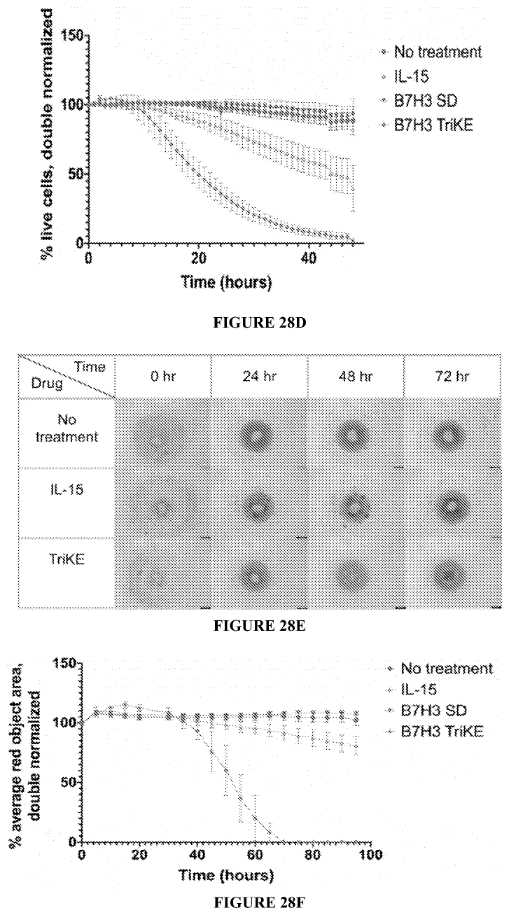

As shown in FIGS. 28A-28F, B7-H3 TriKE induced NK cell killing against HNSCC in real-time imaging assays. Enriched NK cells (N=4) were incubated with Nuclight red-labeled Cal27 at an E:T of 5:1 in different conditions: no treatment or 3 nM IL-15, B7-H3 SD and B7-H3 TriKE for 48 hours in an IncuCyte Zoom imager. Quantifications of percent live cells were done by normalizing hourly counts of red cells to targets alone at t=0. Spheroids of Nuclight red-labeled Cal27 were formed for 72 hours before incubated with enriched NK cells (N=4) at an E:T of 5:1 indifferent conditions: no treatment or 3 nM IL-15, B7-H3 SD and B7-H3 TriKE for 96 hours in an IncuCyte S3 imager. Representative images showing spheroids over time. Quantifications of percent average red object area (live cells) were done by normalizing hourly counts of average red object area to targets alone at t=0. Same set of assays were done with Cal33.

There is a critical need for a targeted therapy that can effectively eliminate HNSCC cells while sparing healthy cells. Here, we described the preclinical study of a TriKE molecule against B7-H3 ligands that are expressed on HNSCC. We have found that treatment with the B7-H3 TriKE effectively induces NK cell degranulation and cytokine production against HNSCC, as well as drives targeted killing of HNSCC in vitro. Ongoing experiments will evaluate the functionality and efficacy of the B7-H3 TriKE in vivo. Future studies will involve investigations of the HNSCC tumor microenvironment, and assessments of the B7-H3 TriKE efficacy in the HNSCC tumor microenvironment in addition to evaluating whether HPV status of HNSCC has any implications on efficacy of the TriKE in the HNSCC tumor microenvironment as previous studies have reported differential NK cell activity in HPV+/−HNSCC tumor microenvironment.

### Example 7

### Efficacy of H7-B3 TriKE in Ovarian Cancer

The efficacy of the H7-B3 TriKE molecules of the invention was assessed in several ovarian cancer cell lines. Unless described otherwise, the methods used are as described in Example 2.

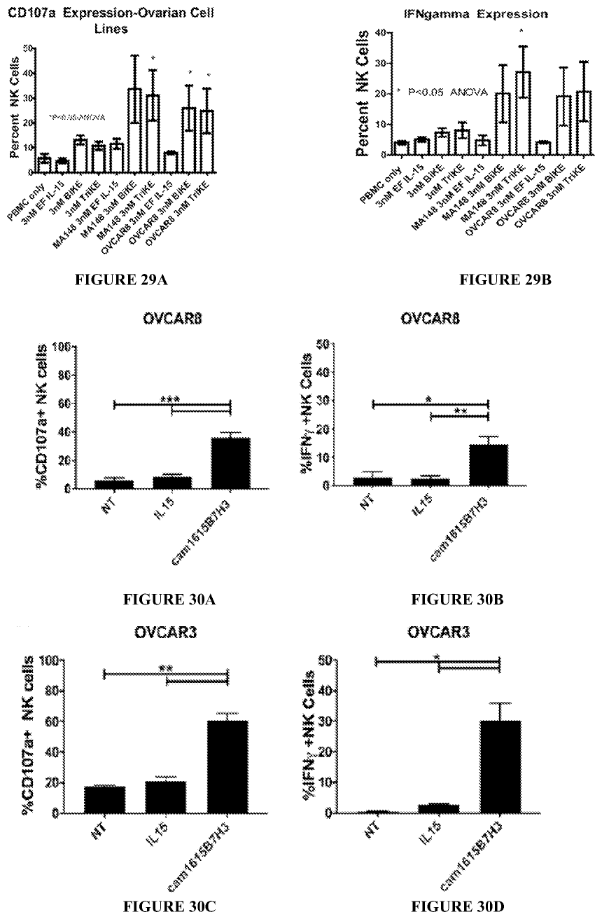

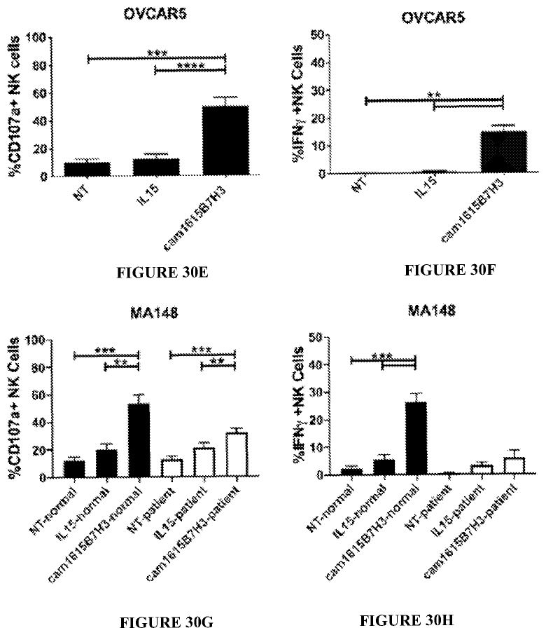

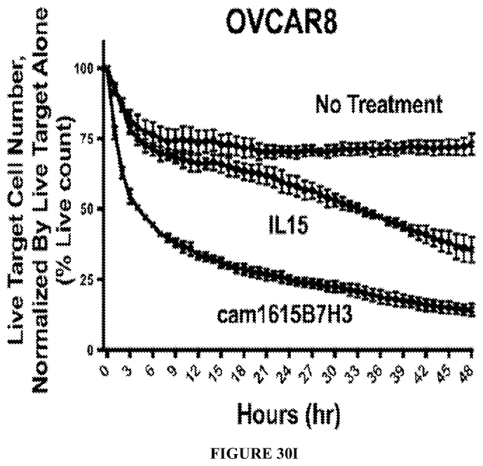

As illustrated in FIGS. 29A-29B and 30A-30I cam1615B7-H3 TriKE exhibits Potent killing of ovarian cancer. The ability of cam1615B7-H3 TriKE to mediate NK cell activity against ovarian cancer cells was evaluated. Ovarian cancer cells used displayed robust B7-H3 expression. Since B7-H3 has been shown to have a role in immune responses, the capacity of the cam1615B7-H3 to induce activity against normal immune cells was evaluated in PBMCs. Flow cytometric assays, allowing for gating on NK cells, determined that the cam1615B7-H3 induced some background degranulation (CD107a) on NK cells in comparison to controls, but this activity was low. No background noise was seen with IFNγ. In contrast, when PBMCs were incubated with a variety of high grade serous ovarian adenocarcinoma cell lines, including OVCAR8, OVCAR3, and OVCAR5, robust NK cell degranulation and intracellular IFNγ production was seen compared to no treatment and rhIL-15 alone (FIGS. 29A-29B and 30A-30F). In order to determine if the individual components of the TriKE could induce NK cell activity on their own, the individual cam16 VHH, IL-15, or anti-B7-H3 scFv components were incubated with PBMCs and OVCAR8 cells and activity was determined. The data clearly shows that individual components do not enhance NK cell activity against OVCAR8 cells. NK cell activity, from normal donor PBMCs and ascites from the peritoneal cavity of ovarian cancer patients at the time of surgery, was assessed against MA-148 cells, another high-grade serous ovarian adenocarcinoma cell line (FIGS. 29A-29B and 30G-30H). Compared to controls, the cam1615B7-H3 TriKE induced robust activity on normal donor NK cells. While NK cell activity from ovarian-cancer-derived ascites samples was decreased, as expected due to alterations in NK cell function driven by the tumor microenvironment and decreases in CD16 expression, the cam1615B7-H3 TriKE induced significantly enhanced NK cell degranulation compared to controls. Finally, killing of ovarian cancer tumor cells (OVCAR8s) was measured dynamically over a two-day period in the presence of enriched NK cells alone (No Treatment), NK cells and rhIL-15 (IL15), and NK cells and the cam1615B7-H3 TriKE (FIG. 30I). In this assay, tumor cells can be tracked with a stably expressed fluorescent protein (NucLight Red) and detection of early apoptosis, used to exclude recent cell death, is mediated by a green fluorescent Caspase3/7 dye. The basic readout provided is the number of tumor cells alive (Red+Green−) normalized to tumor alone at the noted times. As shown, the cam1615B7-H3 TriKE induced robust and rapid tumor killing when compared to controls. This data indicates that the cam1615B7-H3 TriKE potently enhances activity against ovarian cancer cells in vitro. Of note, the cam1615B7-H3 TriKE induced similar degranulation and stronger IFNγ production against ovarian cancer when compared to a potent natural cytotoxicity signal, in the absence of TriKE, induced by K562 cells. Fold NK cell activation against all ovarian cancer cell lines, calculated as activation on PBMC+Tumor+TriKE divided by activation on PBMC+TriKE alone, was higher than activation by the B7-H3-negative Raji line, indicating the B7-H3 specificity of the TriKE.

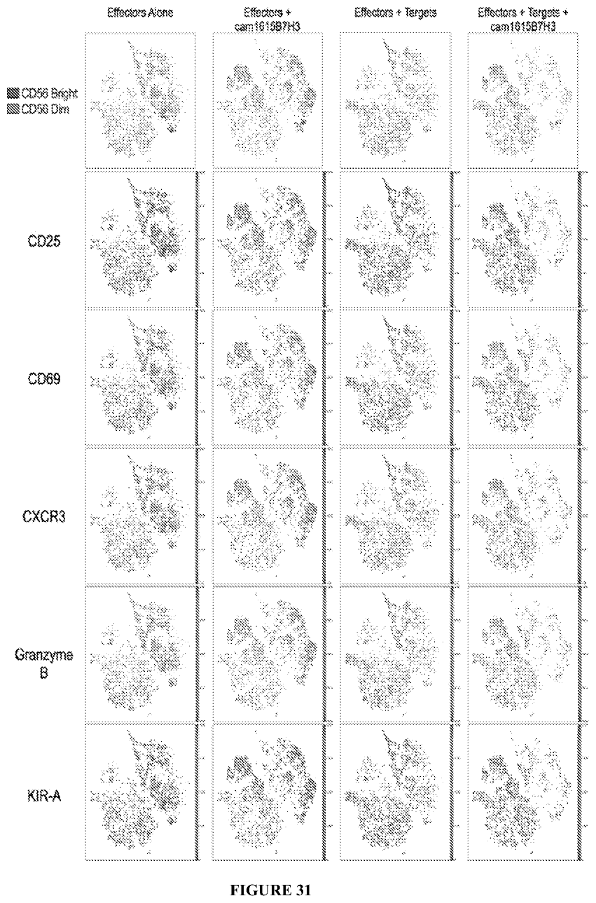

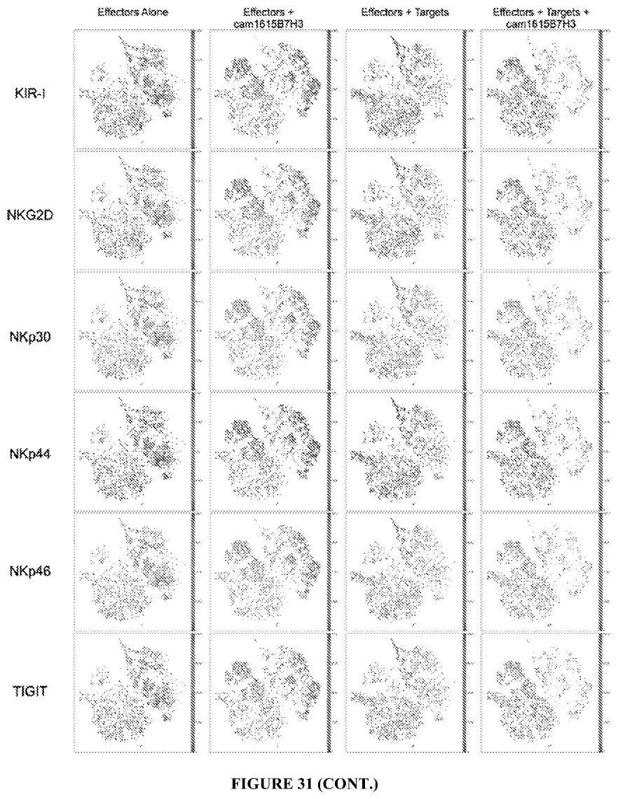

As illustrated in FIG. 31 high dimensional analysis of cam1615B7-H3 TriKE activated cells was performed. To broadly evaluate the phenotypic and functional effects of TriKE activation on NK cells, a custom, 42 parameter, CyTOF (mass cytometry) NK cell targeted panel was used. PBMCs were left untreated, incubated with cam1615B7-H3 TriKE for 24 h, incubated with tumor (OVCAR8s) for 24 h, or incubated with tumor and cam1615B7-H3 TriKE for 24 h. Cells were then stained, fixed, and run on a CyTOF2. Samples (three biologic replicates per condition) were concatenated, and data was visualized with viSNE, which uses all expression information to display localization of individual cells in a 2D plot in order to explore the multidimensional data (FIG. 31). The data indicated no changes in distributions of CD56^{brights} versus CD56^{dims}. Activation markers CD25 and CD69 were both induced with TriKE treatment, as was the chemokine receptor CXCR3. Granzyme B, involved in cytolytic activity of NK cells, was primed on effectors+TriKE, but in the presence of tumor targets (effectors+tumor+TriKE) these Granzyme B high cells disappeared, likely as a cause of ADCC driven specific degranulation. Interestingly, both inhibitory KIR (KIR2DL1, KIR2DL3 and KIR3DL1) and activating KIR (KIR2DS1 and KIR2DS4) were reduced in expression when effectors were exposed to tumor in the presence of TriKE. This also seemed to be the case with NKG2D, but the natural cytotoxicity receptors (NCR: NKp30, NKp44, and NKp46) were less affected. Finally, the inhibitory receptor TIGIT also did not seem as affected. Taken together, this data demonstrates dynamic changes in NK cell phenotype post TriKE mediated activation.

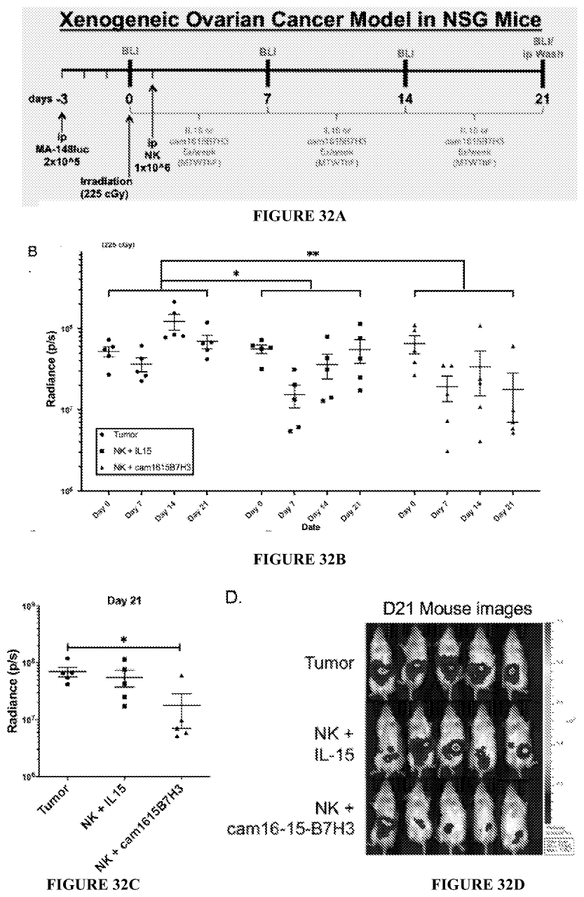

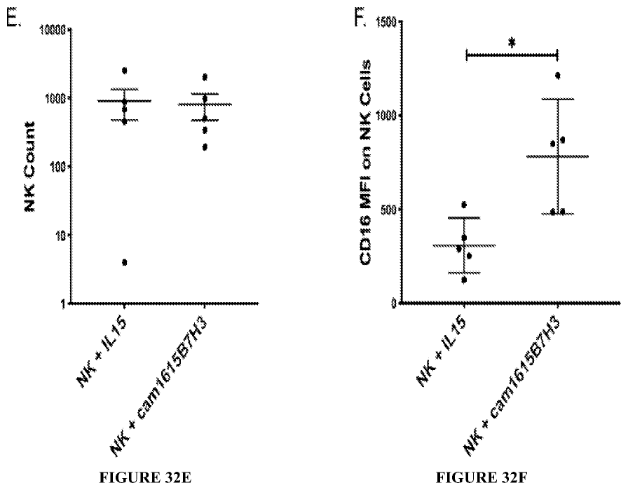

As illustrated in FIGS. 32A-32F, cam1615B7-H3 TriKE mediates anti-tumor activity in vivo. Determination of in vivo activity is a critical step for translation. However, prior to evaluating the ability of the cam1615B7-H3 TriKE to induce function against tumor the potential for toxicity was assessed. To do this NSG mice were irradiated, engrafted with 1 million NK cells, treated with nothing, IL-15, or cam1615B7-H3 for three weeks, and weights were tracked over the course of 90 days post initial treatment. Despite an initial drop in weight in all groups, likely due to the irradiation, no significant differences were seen in the TriKE treated group vs. the controls. This is not surprising given the low toxicity profile of IL-15 and the safety profile of B7-H3. The in vitro data indicates that the cam1615B7-H3 TriKE can potently activate NK cells against a variety of tumors, but to evaluate whether this TriKE has efficacy in a pre-clinical model, a previously described xenogeneic mouse model of ovarian cancer was used (FIG. 32A). In this model, human NK cells and human high grade serous MA-148-luc cells are injected into the peritoneal cavity of NSG mice. Longitudinal analysis of tumor progression showed the cam1615B7-H3 treated mice displayed the lowest tumor progression, when compared to the IL-15 treated or tumor only mice (FIG. 32B). At the time of harvest (day 21), the cam1615B7-H3-treated mice had significantly lower tumor burden than the tumor only group (FIGS. 32C-32D). Peritoneal lavages at this timepoint showed similar human NK cell numbers in the rhIL-15 and cam1615B7-H3 treated groups indicating that the differences in tumor control were not driven by differences in NK cell numbers alone (FIG. 32E). Relevant to the mechanism of action of the cam1615B7-H3 TriKE, TriKE-treated mice had NK cells with higher levels of CD16 expression than IL-15 treated mice (FIG. 32F). PD-1 expression, often associated with exhaustion in immune cells, also had a lower (but not significant) trend of expression in the TriKE-treated vs. the IL-15-treated mice.

### Example 8

### Conclusion and Discussion

Ideal targeted immunotherapeutic interventions for solid tumors will have broad-spectrum recognition of a variety of carcinomas with limited or no on-target off-tumor toxicities. B7-H3 displays these characteristics: it has high expression in a number of tumors and low expression in normal tissues. Targeted antibody-based therapies for B7-H3 are currently being explored in the clinic (NCT04185038, NCT02982941, NCT03406949, NCT03729596, NCT04077866, and NCT02475213). Both the safety profile and efficacy of anti-B7-H3 antibodies in clinical trials thus far have been favorable. Radiolabeled antibodies targeting B7-H3 have been safely administered for at least 10 years. The drug has been deemed safe enough to use intracranially in children. Interestingly, B7-H3 reportedly is expressed on vasculature and stroma fibroblasts, indicating that this antigen could be used to target the tumor vasculature and architecture. A clear correlation exists between high B7-H3 expression and various tumor growth parameters, including fewer tumor-infiltrating lymphocytes, faster cancer progression, and poor clinical outcome in several cancers such as pancreatic ductal adenocarcinoma (PDAC), prostate cancer, ovarian cancer, lung cancer, and clear cell renal carcinoma. Furthermore, natural cytotoxicity against most cancers is usually not enough for endogenous NK cells to keep cancer progression at bay, highlighted by low natural cytotoxicity against most tumor lines tested in this study. Taken together, these studies make a very compelling case for targeting B7-H3.

None of the previous therapeutic approaches, however, combine cytokine signaling and ADCC, two critical components for optimal NK cell immunotherapy. The cam1615B7-H3 protein described here uses that optimal combination. Our data indicates that the cam1615B7-H3 TriKE delivers a specific IL-15 signal to the NK cells, preventing off target toxicities, and also mediates ADCC against a variety of adenocarcinoma cell lines in the ovarian, prostate, and lung cancer settings. This dual mechanism of action allows for enhanced NK cell proliferation, survival, and targeted activation. Our previous studies, comparing TriKEs to bispecific killer engagers (BiKEs) lacking IL-15, have shown that the IL-15 moiety in the TriKE induces NK cell proliferation, survival, increased STAT5 signaling, and enhanced priming. We should note, however, that our in vitro studies show some induction of overall T cell proliferation by the TriKE, albeit minimal in nature when compared to treatment with an equimolar concentration of IL-15. This indicates that, while TriKE is inducing more specificity than monomeric IL-15, it still triggers T cell proliferation at a low level. Interestingly, while overall T cell proliferation when compared to no treatment is increased by the TriKE, proliferation beyond three divisions is actually decreased, and there are no differences in T cell numbers at the end of culture when comparing these two groups. Exploration in more complex models and patients will be needed to fully outline the specificity of the cam1615B7-H3 TriKE and evaluate the impact on T cells and, more importantly, T cell toxicities.

While pre-clinical ovarian cancer mouse model results are encouraging and treated animals had stable disease, the treatment was not curative within this model. This may be due to various factors. Human NK cell donors are variable, a problem that may be solved by breakthroughs in NK cellular products like induced pluripotent stem cell derived NK cells (iNK). Also, the TriKE molecule is small, less than 65 kDa in size, resulting in quick clearance through the kidney and sub-optimal dosing. Different donors might clear at different rates. Alternatively, NK cell exhaustion, either mediated by IL-15 or through strong NK cell activation, could be operant. TriKEs are dependent on targeting CD16 for activation and can be cleaved by the metalloproteinase ADAM17. We and others have previously described low levels of CD16 in the NK cells derived from the ascites of women with ovarian cancer and the MA-148 xenogeneic mouse model mimics this phenomenon. CD16 cleavage might be mediated by either over-activation of the NK cells by the tumor itself or the inflammatory tumor microenvironment, as ADAM17 can be triggered by both activating and cytokine receptors. This is not unique to ovarian cancer, as reduced CD16 expression on NK cells has been described in other tumor settings. Though the CD16 downmodulation may not be seen in every tumor setting, our ascites data indicates that in settings with low CD16 expression the TriKEs can still mediate tumor killing, albeit in a reduced fashion. However, combination with ADAM17 inhibitors, which have been clinically tested for years, or cellular products that have uncleavable CD16 receptors, recently described and currently being clinically tested (NCT04023071), should greatly improve the activity of TriKEs in settings where CD16 is downregulated.

While the majority of immunotherapy modalities focus on checkpoint blockade and T cells, natural killer cells have a number of characteristics that make them ideal candidates for cell-based therapy against solid tumors. These studies focus on a unique biologic platform technology, incorporating IL-15 as a bispecific antibody cross-linker, to drive NK-cell-mediated targeting of a broad spectrum of cancers. TriKEs overcome non-specific mechanisms of natural cytotoxicity by promoting an antigen-specific synapse intended to enhance functional NK cell-mediated killing, activation, and proliferation. The TriKE molecule described in this study targets B7-H3, a member of the B7 costimulatory family of Ig proteins that is overexpressed in a number of solid tumor malignancies. It was found that B7-H3 is a robust target for TriKE molecules, selectively boosting NK-cell in vitro killing of ovarian cancer, prostate cancer, and lung cancer. The IL-15 action is remarkably specific to NK-cell activity with little off-target effects on T cells. This provides the first in vivo xenograft data, supporting the notion that TriKEs can work against solid tumors and supports their future clinical development.

Sequences:
B7-H3 TriKE (cam1615B7-H3) amino acid sequence
(488 residues)
SEQ ID NO: 1
QVQLVESGGGLVQPGGSLRLSCAASGLTFSSYNMGWFRQAPGQGLEAVASITWSGRDTFYADS
VKGRFTISRDNSKNTLYLQMNSLRAEDTAVYYCAANPWPVAAPRSGTYWGQGTLVTVSSPSGQ
AGAAASESLFVSNHAYNWVNVISDLKKIEDLIQSMHIDATLYTESDVHPSCKVTAMKCFLLEL
QVISLESGDASIHDTVENLIILANNSLSSNGNVTESGCKECEELEEKNIKEFLQSFVHIVQMF
INTSEASGGPEDIVMTQSHKFMSTSIGARVSITCKASQDVRTAVAWYQQKPGQSPKLLIYSAS
YRYTGVPDRFTGSGSGTDFTFTISSVQAEDLAVYYCQQHYGTPPWTFGGGTKLEIKEVQLVES
GGGLVKPGGSLKLSCEASRFTFSSYAMSWVRQTPEKRLEWVAAISGGGRYTYYPDSMKGRFTI
SRDNAKNFLYLQMSSLRSEDTAMYYCARHYDGYLDYWGQGTTLTVSS
camCD16
SEQ ID NO: 2
QVQLVESGGGLVQPGGSLRLSCAASGLTFSSYNMGWFRQAPGQGLEAVASITWSGRDTFYADS
VKGRFTISRDNSKNTLYLQMNSLRAEDTAVYYCAANPWPVAAPRSGTYWGQGTLVTVSS
HMA linker
SEQ ID NO: 3
PSGQAGAAASESLFVSNHAY
Wt IL-15
SEQ ID NO: 4
NWVNVISDLKKIEDLIQSMHIDATLYTESDVHPSCKVTAMKCFLLELQVISLESGDASIHDTV
ENLIILANNSLSSNGNVTESGCKECEELEEKNIKEFLQSFVHIVQMFINTS
Linker
SEQ ID NO: 5
EASGGPE
B7-H3 scFv light chain
SEQ ID NO: 6
DIVMTQSHKFMSTSIGARVSITCKASQDVRTAVAWYQQKPGQSPKLLIYSASYRYTGVPDRFT
GSGSGTDFTFTISSVQAEDLAVYYCQQHYGTPPWTFGGGTKLEIK
B7-H3 scFv heavy chain
SEQ ID NO: 7
EVQLVESGGGLVKPGGSLKLSCEASRFTFSSYAMSWVRQTPEKRLEWVAAISGGGRYTYYPDS
MKGRFTISRDNAKNFLYLQMSSLRSEDTAMYYCARHYDGYLDYWGQGTTLTVSS
5′ → 3′ B7-H3 TriKE (cam1615B7-H3) DNA sequence
(1,464 residues)
SEQ ID NO: 8
caggtgcagctggtggagtctgggggaggcttggtgcagcctgggggctctctgagactctcc
tgtgcagcctctggcctcaccttcagtagctataacatgggctggttccgccaggctccaggg
caaggccttgaggctgtagcatctattacctggagtggtcgggacacattctatgcagactcc
gtgaagggccgattcaccatctccagagacaactccaagaacactctctatctgcaaatgaac
agcctgcgcgcggaggacacggccgtttattattgtgctgcaaacccctggccagtggcggcg
ccacgtagtggcacctactggggccaagggaccctggtcaccgtctcctcaccgtctggtcag
gctggtgctgctgctagcgaatctctgttcgtttctaaccacgcttacaactgggtgaatgta
ataagtgatttgaaaaaaattgaagatcttattcaatctatgcatattgatgctactttatat
acggaaagtgatgttcaccccagttgcaaagtaacagcaatgaagtgctttctcttggagtta
caagttatttcacttgagtccggagatgcaagtattcatgatacagtagaaaatctgatcatc
ctagcaaacaacagtttgtcttctaatgggaatgtaacagaatctggatgcaaagaatgtgag
gaactggaggaaaaaaatattaaagaatttttgcagagttttgtacatattgtccaaatgttc
atcaacacttctgaagcttccggaggtcccgaggatattgtgatgacccagagccataaattt
atgagcaccagcattggcgcgcgcgtgagcattacctgcaaagcgagccaggatgtgcgcacc
gcggtggcgtggtatcagcagaaaccgggccagagcccgaaactgctgatttatagcgcgagc
tatcgctataccggcgtgccggatcgctttaccggcagcggcagcggcaccgattttaccttt
accattagcagcgtgcaggcggaagatctggcggtgtattattgccagcagcattatggcacc
ccgccgtggacctttggcggcggcaccaaactggaaattaaagaagtgcagctggtggaaagc
ggcggcggcctggtgaaaccgggcggcagcctgaaactgagctgcgaagcgagccgctttacc
tttagcagctatgcgatgagctgggtgcgccagaccccggaaaaacgcctggaatgggtggcg
gcgattagcggggggccgctatacctattatccggatagcatgaaaggccgctttaccattag
ccgcgataacgcgaaaaactttctgtatctgcagatgagcagcctgcgcagcgaagataccgc
gatgtattattgcgcgcgccattatgatggctatctggattattggggccagggcaccaccct
gaccgtgagcagc
camCD16
SEQ ID NO: 9
caggtgcagctggtggagtctgggggaggcttggtgcagcctgggggctctctgagactctcc
tgtgcagcctctggcctcaccttcagtagctataacatgggctggttccgccaggctccaggg
caaggccttgaggctgtagcatctattacctggagtggtcgggacacattctatgcagactcc
gtgaagggccgattcaccatctccagagacaactccaagaacactctctatctgcaaatgaac
agcctgcgcgcggaggacacggccgtttattattgtgctgcaaacccctggccagtggcggcg
ccacgtagtggcacctactggggccaagggaccctggtcaccgtctcctca
HMA Linker
SEQ ID NO: 10
ccgtctggtcaggctggtgctgctgctagcgaatctctgttcgtttctaaccacgcttac
wtIL-15
SEQ ID NO: 11
aactgggtgaatgtaataagtgatttgaaaaaaattgaagatcttattcaatctatgcatatt
gatgctactttatatacggaaagtgatgttcaccccagttgcaaagtaacagcaatgaagtgc
tttctcttggagttacaagttatttcacttgagtccggagatgcaagtattcatgatacagta
gaaaatctgatcatcctagcaaacaacagtttgtcttctaatgggaatgtaacagaatctgga
tgcaaagaatgtgaggaactggaggaaaaaaatattaaagaatttttgcagagttttgtacat
attgtccaaatgttcatcaacacttct
linker
SEQ ID NO: 12
gaagcttccggaggtcccgag
B7-H3 scFv Light Chain
SEQ ID NO: 13
gatattgtgatgacccagagccataaatttatgagcaccagcattggcgcgcgcgtgagcatt
acctgcaaagcgagccaggatgtgcgcaccgcggtggcgtggtatcagcagaaaccgggccag
agcccgaaactgctgatttatagcgcgagctatcgctataccggcgtgccggatcgctttacc
ggcagcggcagcggcaccgattttacctttaccattagcagcgtgcaggcggaagatctggcg
gtgtattattgccagcagcattatggcaccccgccgtggacctttggcggcggcaccaaactg
gaaattaaa
B7-H3 scFv Heavy Chain
SEQ ID NO: 14
gaagtgcagctggtggaaagcggcggcggcctggtgaaaccgggcggcagcctgaaactgagc
tgcgaagcgagccgctttacctttagcagctatgcgatgagctgggtgcgccagaccccggaa
aaacgcctggaatgggtggcggcgattagcggcggcggccgctatacctattatccggatagc
atgaaaggccgctttaccattagccgcgataacgcgaaaaactttctgtatctgcagatgagc
agcctgcgcagcgaagataccgcgatgtattattgcgcgcgccattatgatggctatctggat
tattggggccagggcaccaccctgaccgtgagcagc
Linkseq 16 Linker
SEQ ID NO: 15
SGGGGSGGGGSGGGGSGGGGSG
Mammalian linker
SEQ ID NO: 16
GSTSGSGKPGSGEGSTKG
IL-15 N72D mutation
SEQ ID NO: 17
NWVNVISDLKKIEDLIQSMHIDATLYTESDVHPSCKVTAMKCFLLELQVISLESGDASIHDTV
ENLIILANDSLSSNGNVTESGCKECEELEEKNIKEFLQSFVHIVQMFINTS
IL-15 N72A mutation
SEQ ID NO: 18
NWVNVISDLKKIEDLIQSMHIDATLYTESDVHPSCKVTAMKCFLLELQVISLESGDASIHDTV
ENLIILANASLSSNGNVTESGCKECEELEEKNIKEFLQSFVHIVQMFINTS
human CD16 amino acid
SEQ ID NO: 19
MEVQLVESGGGVVRPGGSLRLSCAASGFTFDDYGMSWVRQAPGKGLEWVSGINWNGGSTGYAD
SVKGRFTISRDNAKNSLYLQMNSLRAEDTAVYYCARGRSLLFDYWGQGTLVTVSRGGGGSGGG
GSGGGGSSELTQDPAVSVALGQTVRITCQGDSLRSYYASWYQQKPGQAPVLVIYGKNNRPSGI
PDRFSGSSSGNTASLTITGAQAEDEADYYCNSRDSSGNHVVFGGGTKLTVL
human CD16 DNA
SEQ ID NO: 20
atggaagtgcagctggtggaaagcggcggcggcgtggtgcgcccgggcggcagcctgcgcctg
agctgcgcggcgagcggctttacctttgatgattatggcatgagctgggtgcgccaggcgccg
ggcaaaggcctggaatgggtgagcggcattaactggaacggcggcagcaccggctatgcggat
agcgtgaaaggccgctttaccattagccgcgataacgcgaaaaacagcctgtatctgcagatg
aacagcctgcgcgcggaagataccgcggtgtattattgcgcgcgcggccgcagcctgctgttt
gattattggggccagggcaccctggtgaccgtgagccgcggcggcggcggcagcggcggcggc
ggcagcggcggcggcggcagcagcgaactgacccaggatccggcggtgagcgtggcgctgggc
cagaccgtgcgcattacctgccagggcgatagcctgcgcagctattatgcgagctggtatcag
cagaaaccgggccaggcgccggtgctggtgatttatggcaaaaacaaccgcccgagcggcatt
ccggatcgctttagcggcagcagcagcggcaacaccgcgagcctgaccattaccggcgcgcag
gcggaagatgaagcggattattattgcaacagccgcgatagcagcggcaaccatgtggtgttt
ggcggcggcaccaaactgaccgtgctg
HLE sequence 1
SEQ ID NO: 21
DKTHTCPPCPAPELLGGPSVFLFPPKPKDTLMISRTPEVTCVWDVSHEDPEVKENWYVDGVEV
HNAKTKPCEEQYNSYRCVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGGGGSGGGGSGGGGSGGGGSGGGGSGGGG
SDKTHTCPPCPAPELLGGPSVFLFPPKPKDTLMISRTPEVTCWVDVSHEDPEVKFNWYDGVEV
HNAKTKPCEEQYNSTYRCVSVLTVLHQDWLNGKEYKCKVSNKALPAPIEKTISKAKGQPREPQ
VYTLPPSREEMKNQVSLTCLVKGFYPSDIAVEWESNGQPENNYKTTPPVLDSDGSFFLYSKLT
VDKSRWQQGNVFSCSVMHEALHNHYTQKSLSLSP
HLE sequence 2
SEQ ID NO: 22
DKTHTCPPCPAPELLGGPSVFLFPPKPKDTLMISRTPEVTCVWDVSHEDPEVKFNWYVDGVEV
HNAKTKPCEEQYNSTYRCVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGKGGGGSGGGGSGGGGSGGGGSGGGGSG
GGGSDKTHTCPPCPAPELLGGPSVFLFPPKPKDTLMISRTPEVTCWVDVSHEDPEVKFNWYVD
GVEVHNAKTKPCEEQYNSTYRCVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
HLE sequence 3
SEQ ID NO: 23
DKTHTCPPCPAPELLGGPSVFLFPPKPKDTLMISRTPEVTCWVDVSHEDPEVKENWYVDGVEV
HNAKTKPCEEQYGSTYRCVSVLTVLHQDWLNGKEYKCKVSNKALPAPIEKTISKAKGQPREPQ
VYTLPPSRKEMTKNQVSLTCLVKGFYPSDIAVEWESNGQPENNYKTTPPVLKSDGSFFLYSKL
TVDKSRWQQGNVFSCSVMHEALHNHYTQKSLSLSPGK
HLE sequence 4
SEQ ID NO: 24
DKTHTCPPCPAPELLGGPSVFLFPPKPKDTLMISRTPEVTCVWDVSHEDPEVKFNWYVDGVEV
HNAKTKPCEEQYGSTYRCVSVLTVLHQDWLNGKEYKCKVSNKALPAPIEKTISKAKGQPREPQ
VYTLPPSREEMTKNQVSLTCLVKGFYPSDIAVEWESNGQPENNYDTTPPVLDSDGSFFLYSDL
TVDKSRWQQGNVFSCSVMHEALHNHYTQKSLSLSPGK
HLE sequence 5
SEQ ID NO: 25
DKTHTCPPCPAPELLGGPSVFLFPPKPKDTLMISRTPEVTCWVDVSHEDPEVKFNWYVDGVEV
HNAKTKPCEEQYGSTYRCVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGKGGGGSGGGGSGGGGSGGGGSGGGGSG
GGGSDKTHTCPPCPAPELLGGPSVFLFPPKPKDTLMISRTPEVTCVWDVSHEDPEVKFNWYVD
GVEVHNAKTKPCEEQYGSTYRCVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Fc region 1
SEQ ID NO: 26
DKTHTCPPCPAPELLGGPSVFLFPPKPKDTLMISRTPEVTCVWDVSHEDPEVKFNWYVDGVEV
HNAKTKPCEEQYNSYRCVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSP
Fc region 2
SEQ ID NO: 27
DKTHTCPPCPAPELLGGPSVFLFPPKPKDTLMISRTPEVTCWVDVSHEDPEVKFNWYDGVEVH
NAKTKPCEEQYNSTYRCVSVLTVLHQDWLNGKEYKCKVSNKALPAPIEKTISKAKGQPREPQV
YTLPPSREEMKNQVSLTCLVKGFYPSDIAVEWESNGQPENNYKTTPPVLDSDGSFFLYSKLTV
DKSRWQQGNVFSCSVMHEALHNHYTQKSLSLSP
Fc region 3
SEQ ID NO: 28
DKTHTCPPCPAPELLGGPSVFLFPPKPKDTLMISRTPEVTCVWDVSHEDPEVKENWYVDGVEV
HNAKTKPCEEQYNSTYRCVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK
Fc region 4
SEQ ID NO: 29
DKTHTCPPCPAPELLGGPSVFLFPPKPKDTLMISRTPEVTCWVDVSHEDPEVKFNWYVDGVEV
HNAKTKPCEEQYNSTYRCVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK
Fc region 5
SEQ ID NO: 30
DKTHTCPPCPAPELLGGPSVFLFPPKPKDTLMISRTPEVTCWVDVSHEDPEVKFNWYVDGVEV
HNAKTKPCEEQYGSTYRCVSVLTVLHQDWLNGKEYKCKVSNKALPAPIEKTISKAKGQPREPQ
VYTLPPSRKEMTKNQVSLTCLVKGFYPSDIAVEWESNGQPENNYKTTPPVLKSDGSFFLYSKL
TVDKSRWQQGNVFSCSVMHEALHNHYTQKSLSLSPGK
Fc region 6
SEQ ID NO: 31
DKTHTCPPCPAPELLGGPSVFLFPPKPKDTLMISRTPEVTCVWDVSHEDPEVKENWYVDGVEV
HNAKTKPCEEQYGSTYRCVSVLTVLHQDWLNGKEYKCKVSNKALPAPIEKTISKAKGQPREPQ
VYTLPPSREEMTKNQVSLTCLVKGFYPSDIAVEWESNGQPENNYDTTPPVLDSDGSFFLYSDL
TVDKSRWQQGNVFSCSVMHEALHNHYTQKSLSLSPGK
Fc region 7
SEQ ID NO: 32
DKTHTCPPCPAPELLGGPSVFLFPPKPKDTLMISRTPEVTCWVDVSHEDPEVKFNWYVDGVEV
HNAKTKPCEEQYGSTYRCVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK
Fc region 8
SEQ ID NO: 33
DKTHTCPPCPAPELLGGPSVFLFPPKPKDTLMISRTPEVTCVWDVSHEDPEVKFNWYVDGVEV
HNAKTKPCEEQYGSTYRCVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK
scFc linker 1
SEQ ID NO: 34
GGGGSGGGGSGGGGSGGGGSGGGGSGGGGS
scFc linker 2
SEQ ID NO: 35
SSGGGGSGGGGSGGGGS
20 amino acid segment
SEQ ID NO: 36
PSGQAGAAASESLFVSNHAY
seven amino acid linker
SEQ ID NO: 37
EASGGPE

Although the invention has been described with reference to the above examples, it will be understood that modifications and variations are encompassed within the spirit and scope of the invention. Accordingly, the invention is limited only by the following claims.

1. An isolated nucleic acid sequence as set forth in SEQ ID NO:13 or 14 or a sequence having 90% identity thereto.

2. A protein encoded by a nucleic acid sequence of claim 1.

3. The protein of claim 2, wherein the amino acid sequence is selected from SEQ ID NO:6 or 7.

4. A fusion protein comprising the amino acid sequence set forth in SEQ ID NO:6 and 7, operably linked to each other in either orientation.

5. The fusion protein of claim 4, wherein the protein comprises SEQ ID NO:6 and 7, in direct linkage between the C-terminus of SEQ ID NO:6 and the N-terminus of SEQ ID NO: 7.

6. The fusion protein of claim 4, wherein the protein comprises SEQ ID NO:7 and 6, in direct linkage between the C-terminus of SEQ ID NO:7 and the N-terminus of SEQ ID NO: 6.

7. A fusion protein comprising the sequence set forth in SEQ ID NO:1 and sequences having 90% or greater identity to SEQ ID NO:1.

8. A fusion protein comprising in operably linkage, SEQ ID NO:2 or 19; 4, 17, or 18; 6 and 7 or 7 and 6.

9. The fusion protein of claim 8, wherein SEQ ID NO:2 or 19 and 4, 17 or 18 are linked by SEQ ID NO:3 or SEQ ID NO:15.

10. The fusion protein of claim 8, wherein SEQ ID NO:4, 17 or 18 and 6 or 7 are linked by SEQ ID NO:5 or SEQ ID NO:16.

11. The fusion protein of claim 8, wherein SEQ ID NO:6 and 7 are in operable linkage in either orientation.

12. The fusion protein of claim 8, further comprising a half-life extending (HLE) molecule.

13. The fusion protein of claim 12, wherein the HLE molecule is a Fc or a scFc antibody fragment comprising any one of SEQ ID NOs:21-25.

14. The fusion protein of claim 8, wherein SEQ ID NO:4 has an N72 substitution.

15. The fusion protein of claim 14, wherein the N72 mutation is N72A or N72D.

16. The fusion protein of claim 15, wherein the protein is set forth in SEQ ID NO:17 or 18.

17. An isolated nucleic acid sequence encoding the fusion protein of claim 7.

18. The isolated nucleic acid sequence of claim 17, wherein the sequence is SEQ ID NO:8.

19. A method of treating cancer in a subject comprising administering to the subject a fusion protein of claim 4, thereby treating the cancer.

20. The method of claim 19, wherein the cancer is selected from non-small lung cancer, cutaneous squamous cell carcinoma, pancreatic cancer, primary hepatocellular carcinoma, colorectal carcinoma, clear cell renal carcinoma or breast cancer.

21. A fusion protein comprising SEQ ID NO:19, SEQ ID NO:17 or 18 and SEQ ID NO:6 and 7 in either orientation.

22. The fusion protein of claim 21, wherein SEQ ID NO:19 is operably linked to SEQ ID NO:17 or 18 by a linker of SEQ ID NO:3 or 15.

23. The fusion protein of claim 21, wherein SEQ ID NO:17 or 18 is operably linked to SEQ 6 and 7, in either orientation by a linker of SEQ ID NO:5 or 16.

24. The fusion protein of claim 21, further comprising a half-life extending (HLE) molecule.

25. The fusion protein of claim 24, wherein the HLE molecule is a Fc or a scFc antibody fragment comprising any one of SEQ ID NOs:21-25.

26. A pharmaceutical composition comprising a therapeutically effective amount of a fusion protein comprising the amino acid sequence of SEQ ID NO:1 or a sequence having 90% or greater identity to SEQ ID NO:1 and a pharmaceutically acceptable carrier.

27. A method of treating cancer in a subject comprising administering to the subject the pharmaceutical composition of claim 26.

28-38. (canceled)


SEQUENCE LISTING (37 sequences)

SEQ ID NO: 1 - protein, 488 aa, synthetic construct
  REGION 1..488: note = B7H3 TriKE (cam1615B7H3) amino acid sequence (488 residues)
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSPSGQAGAAASESLFVSNHAYNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEASGGPEDIVMTQSHKFMSTSIGARVSITCKASQDVRTAVAWYQQKPGQSPKLLIYSASYRYTGVPDRFTGSGSGTDFTFTISSVQAEDLAVYYCQQHYGTPPWTFGGGTKLEIKEVQLVESGGGLVKPGGSLKLSCEASRFTFSSYAMSWVRQTPEKRLEWVAAISGGGRYTYYPDSMKGRFTISRDNAKNFLYLQMSSLRSEDTAMYYCARHYDGYLDYWGQGTTLTVSS

SEQ ID NO: 2 - protein, 122 aa, synthetic construct
  REGION 1..122: note = camCD16
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSS

SEQ ID NO: 3 - protein, 20 aa, synthetic construct
  REGION 1..20: note = HMA linker
PSGQAGAAASESLFVSNHAY

SEQ ID NO: 4 - protein, 114 aa, synthetic construct
  REGION 1..114: note = Wt IL-15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 7 aa, synthetic construct
  REGION 1..7: note = Linker
EASGGPE

SEQ ID NO: 6 - protein, 108 aa, synthetic construct
  REGION 1..108: note = B7H3 scFv light chain
DIVMTQSHKFMSTSIGARVSITCKASQDVRTAVAWYQQKPGQSPKLLIYSASYRYTGVPDRFTGSGSGTDFTFTISSVQAEDLAVYYCQQHYGTPPWTFGGGTKLEIK

SEQ ID NO: 7 - protein, 117 aa, synthetic construct
  REGION 1..117: note = B7H3 scFv heavy chain
EVQLVESGGGLVKPGGSLKLSCEASRFTFSSYAMSWVRQTPEKRLEWVAAISGGGRYTYYPDSMKGRFTISRDNAKNFLYLQMSSLRSEDTAMYYCARHYDGYLDYWGQGTTLTVSS

SEQ ID NO: 8 - DNA, 1464 bp, synthetic construct
  misc_feature 1..1464: note = 5 prime to 3 prime B7H3 TriKE (cam1615B7H3) DNA sequence (1,464 residues)
  source 1..1464: mol_type = other DNA
caggtgcagctggtggagtctgggggaggcttggtgcagcctgggggctctctgagactctcctgtgcagcctctggcctcaccttcagtagctataacatgggctggttccgccaggctccagggcaaggccttgaggctgtagcatctattacctggagtggtcgggacacattctatgcagactccgtgaagggccgattcaccatctccagagacaactccaagaacactctctatctgcaaatgaacagcctgcgcgcggaggacacggccgtttattattgtgctgcaaacccctggccagtggcggcgccacgtagtggcacctactggggccaagggaccctggtcaccgtctcctcaccgtctggtcaggctggtgctgctgctagcgaatctctgttcgtttctaaccacgcttacaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctgaagcttccggaggtcccgaggatattgtgatgacccagagccataaatttatgagcaccagcattggcgcgcgcgtgagcattacctgcaaagcgagccaggatgtgcgcaccgcggtggcgtggtatcagcagaaaccgggccagagcccgaaactgctgatttatagcgcgagctatcgctataccggcgtgccggatcgctttaccggcagcggcagcggcaccgattttacctttaccattagcagcgtgcaggcggaagatctggcggtgtattattgccagcagcattatggcaccccgccgtggacctttggcggcggcaccaaactggaaattaaagaagtgcagctggtggaaagcggcggcggcctggtgaaaccgggcggcagcctgaaactgagctgcgaagcgagccgctttacctttagcagctatgcgatgagctgggtgcgccagaccccggaaaaacgcctggaatgggtggcggcgattagcggcggcggccgctatacctattatccggatagcatgaaaggccgctttaccattagccgcgataacgcgaaaaactttctgtatctgcagatgagcagcctgcgcagcgaagataccgcgatgtattattgcgcgcgccattatgatggctatctggattattggggccagggcaccaccctgaccgtgagcagc

SEQ ID NO: 9 - DNA, 366 bp, synthetic construct
  misc_feature 1..366: note = camCD16
  source 1..366: mol_type = other DNA
caggtgcagctggtggagtctgggggaggcttggtgcagcctgggggctctctgagactctcctgtgcagcctctggcctcaccttcagtagctataacatgggctggttccgccaggctccagggcaaggccttgaggctgtagcatctattacctggagtggtcgggacacattctatgcagactccgtgaagggccgattcaccatctccagagacaactccaagaacactctctatctgcaaatgaacagcctgcgcgcggaggacacggccgtttattattgtgctgcaaacccctggccagtggcggcgccacgtagtggcacctactggggccaagggaccctggtcaccgtctcctca

SEQ ID NO: 10 - DNA, 60 bp, synthetic construct
  misc_feature 1..60: note = HMA Linker
  source 1..60: mol_type = other DNA
ccgtctggtcaggctggtgctgctgctagcgaatctctgttcgtttctaaccacgcttac

SEQ ID NO: 11 - DNA, 342 bp, synthetic construct
  misc_feature 1..342: note = wtIL-15
  source 1..342: mol_type = other DNA
aactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 12 - DNA, 21 bp, synthetic construct
  misc_feature 1..21: note = linker
  source 1..21: mol_type = other DNA
gaagcttccggaggtcccgag

SEQ ID NO: 13 - DNA, 324 bp, synthetic construct
  misc_feature 1..324: note = B7H3 scFv Light Chain
  source 1..324: mol_type = other DNA
gatattgtgatgacccagagccataaatttatgagcaccagcattggcgcgcgcgtgagcattacctgcaaagcgagccaggatgtgcgcaccgcggtggcgtggtatcagcagaaaccgggccagagcccgaaactgctgatttatagcgcgagctatcgctataccggcgtgccggatcgctttaccggcagcggcagcggcaccgattttacctttaccattagcagcgtgcaggcggaagatctggcggtgtattattgccagcagcattatggcaccccgccgtggacctttggcggcggcaccaaactggaaattaaa

SEQ ID NO: 14 - DNA, 351 bp, synthetic construct
  misc_feature 1..351: note = B7H3 scFv Heavy Chain
  source 1..351: mol_type = other DNA
gaagtgcagctggtggaaagcggcggcggcctggtgaaaccgggcggcagcctgaaactgagctgcgaagcgagccgctttacctttagcagctatgcgatgagctgggtgcgccagaccccggaaaaacgcctggaatgggtggcggcgattagcggcggcggccgctatacctattatccggatagcatgaaaggccgctttaccattagccgcgataacgcgaaaaactttctgtatctgcagatgagcagcctgcgcagcgaagataccgcgatgtattattgcgcgcgccattatgatggctatctggattattggggccagggcaccaccctgaccgtgagcagc

SEQ ID NO: 15 - protein, 22 aa, synthetic construct
  REGION 1..22: note = Linkseq16 Linker
SGGGGSGGGGSGGGGSGGGGSG

SEQ ID NO: 16 - protein, 18 aa, synthetic construct
  REGION 1..18: note = Mammalian linker
GSTSGSGKPGSGEGSTKG

SEQ ID NO: 17 - protein, 114 aa, synthetic construct
  REGION 1..114: note = IL-15 N72D mutation
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 18 - protein, 114 aa, synthetic construct
  REGION 1..114: note = IL-15 N72A mutation
NWVNVISDLKKIEDLIQSMHIDATLYTESDVHPSCKVTAMKCFLLELQVISLESGDASIHDTVENLIILANASLSSNGNVTESGCKECEELEEKNIKEFLQSFVHIVQMFINTS

SEQ ID NO: 19 - protein, 240 aa, synthetic construct
  REGION 1..240: note = human CD16 amino acid
MEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 20 - DNA, 720 bp, synthetic construct
  misc_feature 1..720: note = human CD16 DNA
  source 1..720: mol_type = other DNA
atggaagtgcagctggtggaaagcggcggcggcgtggtgcgcccgggcggcagcctgcgcctgagctgcgcggcgagcggctttacctttgatgattatggcatgagctgggtgcgccaggcgccgggcaaaggcctggaatgggtgagcggcattaactggaacggcggcagcaccggctatgcggatagcgtgaaaggccgctttaccattagccgcgataacgcgaaaaacagcctgtatctgcagatgaacagcctgcgcgcggaagataccgcggtgtattattgcgcgcgcggccgcagcctgctgtttgattattggggccagggcaccctggtgaccgtgagccgcggcggcggcggcagcggcggcggcggcagcggcggcggcggcagcagcgaactgacccaggatccggcggtgagcgtggcgctgggccagaccgtgcgcattacctgccagggcgatagcctgcgcagctattatgcgagctggtatcagcagaaaccgggccaggcgccggtgctggtgatttatggcaaaaacaaccgcccgagcggcattccggatcgctttagcggcagcagcagcggcaacaccgcgagcctgaccattaccggcgcgcaggcggaagatgaagcggattattattgcaacagccgcgatagcagcggcaaccatgtggtgtttggcggcggcaccaaactgaccgtgctg

SEQ ID NO: 21 - protein, 475 aa, synthetic construct
  REGION 1..475: note = HLE sequence 1
DKTHTCPPCPAPELLGGPSVFLFPPKPKDTLMISRTPEVTCVWDVSHEDPEVKFNWYVDGVEVHNAKTKPCEEQYNSYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCWVDVSHEDPEVKFNWYDGVEVHNAKTKPCEEQYNSTYRCVSVLTVLHQDWLNGKEYKCKVSNKALPAPIEKTISKAKGQPREPQVYTLPPSREEMKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 22 - protein, 482 aa, synthetic construct
  REGION 1..482: note = HLE sequence 2
DKTHTCPPCPAPELLGGPSVFLFPPKPKDTLMISRTPEVTCVWDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCWVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 226 aa, synthetic construct
  REGION 1..226: note = HLE sequence 3
DKTHTCPPCPAPELLGGPSVFLFPPKPKDTLMISRTPEVTCVWDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 24 - protein, 226 aa, synthetic construct; identical to SEQ ID NO: 23
  REGION 1..226: note = HLE sequence 4

SEQ ID NO: 25 - protein, 482 aa, synthetic construct
  REGION 1..482: note = HLE sequence 5
DKTHTCPPCPAPELLGGPSVFLFPPKPKDTLMISRTPEVTCWVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVWDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 223 aa, synthetic construct
  REGION 1..223: note = Fc region 1
DKTHTCPPCPAPELLGGPSVFLFPPKPKDTLMISRTPEVTCVWDVSHEDPEVKFNWYVDGVEVHNAKTKPCEEQYNSYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 27 - protein, 222 aa, synthetic construct
  REGION 1..222: note = Fc region 2
DKTHTCPPCPAPELLGGPSVFLFPPKPKDTLMISRTPEVTCWVDVSHEDPEVKFNWYDGVEVHNAKTKPCEEQYNSTYRCVSVLTVLHQDWLNGKEYKCKVSNKALPAPIEKTISKAKGQPREPQVYTLPPSREEMKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 28 - protein, 226 aa, synthetic construct
  REGION 1..226: note = Fc region 3
DKTHTCPPCPAPELLGGPSVFLFPPKPKDTLMISRTPEVTCVWDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 226 aa, synthetic construct
  REGION 1..226: note = Fc region 4
DKTHTCPPCPAPELLGGPSVFLFPPKPKDTLMISRTPEVTCWVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 226 aa, synthetic construct
  REGION 1..226: note = Fc region 5
DKTHTCPPCPAPELLGGPSVFLFPPKPKDTLMISRTPEVTCWVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 31 - protein, 226 aa, synthetic construct; identical to SEQ ID NO: 23
  REGION 1..226: note = Fc region 6

SEQ ID NO: 32 - protein, 226 aa, synthetic construct
  REGION 1..226: note = Fc region 7
DKTHTCPPCPAPELLGGPSVFLFPPKPKDTLMISRTPEVTCWVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 226 aa, synthetic construct
  REGION 1..226: note = Fc region 8
DKTHTCPPCPAPELLGGPSVFLFPPKPKDTLMISRTPEVTCVWDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 30 aa, synthetic construct
  REGION 1..30: note = scFc linker 1
GGGGSGGGGSGGGGSGGGGSGGGGSGGGGS

SEQ ID NO: 35 - protein, 17 aa, synthetic construct
  REGION 1..17: note = scFc linker 2
SSGGGGSGGGGSGGGGS

SEQ ID NO: 36 - protein, 20 aa, synthetic construct
  REGION 1..20: note = 20 amino acid (aa) segment
PSGQAGAAASESLFVSNHAY

SEQ ID NO: 37 - protein, 7 aa, synthetic construct
  REGION 1..7: note = linker
EASGGPE